SEQUENCE LISTING (5 sequences)

SEQ ID NO: 1 - DNA, 70 bp, homo sapiens
  J_segment (33)..(44): J-segment of barcode
  misc_feature (33)..(40): n is a, c, g, or t
tttttttaagcagtggtatcaacgcagagtacnnnnnnnntttttttttttttttttttttttttttttt

SEQ ID NO: 2 - DNA, 33 bp, homo sapiens
aagcagtggtatcaacgcagagtgaatrgrgrg

SEQ ID NO: 3 - DNA, 23 bp, homo sapiens
aagcagtggtatcaacgcagagt

SEQ ID NO: 4 - DNA, 66 bp, homo sapiens
aatgatacggcgaccaccgagatctacacgcctgtccgcggaagcagtggtatcaacgcagagtac

SEQ ID NO: 5 - DNA, 37 bp, homo sapiens
gcctgtccgcggaagcagtggtatcaacgcagagtac

(19) United States
(12) Patent Application Publication
(10) Pub. No.: US 2023/0183796 A1
(43) Pub. Date: Jun. 15, 2023

(54) AN INTEGRATED DIELECTROPHORESIS-TRAPPING AND NANOWELL TRANSFER APPROACH TO ENABLE DOUBLE-SUB-POISSON SINGLE-CELL RNA-SEQUENCING

(71) Applicant: Yale University, New Haven, CT (US)

(72) Inventors: Zhiliang Bai, New Haven, CT (US); Rong Fan, Cheshire, CT (US)

(21) Appl. No.: 17/999,324

(22) PCT Filed: May 20, 2021
(86) PCT No.: PCT/US21/33378

§ 371 Date: Nov. 18, 2022

Related U.S. Application Data
(60) Provisional application No. 63/027,582, filed on May 20, 2020.

(51) Int. Cl. C12Q 1/6869; B01L 3/00

(52) U.S. Cl. CPC C12Q 1/6869; B01L 3/502761; B01L 2400/0424; B01L 2200/0668; B01L 2300/0829; C12Q 2600/16; C12Q 1/6844

(57) ABSTRACT

The present invention provides systems and methods for single-cell RNA sequencing. Embodiments of the methods of the present invention include the steps of: aligning a microwell array on top of a dielectrophoresis (DEP) single-cell-trapping nanowell array; loading a plurality of cells into the nanowell; applying electricity to the nanowell array to trap a quanta of cells equal to a quanta of electrode pairs in at least one nanowell of the nanowell array; discontinuing electricity to the nanowell array in order to transfer the loaded cells from the nanowells to the microwells; loading a plurality of barcoded beads into the microwells so that a single bead occupies each cell-loaded microwell; capturing RNA from the cells and retrieving the RNA-loaded beads; and, sequencing the captured RNA.

### CROSS-REFERENCE TO RELATED APPLICATION(S)

This application claims the benefit of priority of U.S. Provisional Patent Application No. 63/027,582, filed May 20, 2020, the contents of which are incorporated by reference herein in their entirety.

### BACKGROUND OF THE INVENTION

Current technologies for high-throughput single-cell RNA-sequencing (scRNA-seq) are based upon stochastic pairing of cells and barcoded beads in nanoliter droplets or wells. It is limited by the mathematical principle of Poisson statistics such that the utilization of either cells or beads or both is no more than ˜33%. Despite the versatile design of microfluidics or microwells for high-yield loading of beads that beats the Poisson limit, subsequent encapsulation of single cells is still determined by stochastic pairing, representing a fundamental limitation in the field of single-cell sequencing.

### SUMMARY OF THE INVENTION

In certain aspects, the present invention provides a method for single-cell RNA sequencing that includes the steps of: aligning a microwell array on top of a dielectrophoresis (DEP) single-cell-trapping nanowell array; loading a plurality of cells into the nanowell; applying electricity to the nanowell array to trap a quanta of cells equal to a quanta of electrode pairs in at least one nanowell of the nanowell array; discontinuing electricity to the nanowell array in order to transfer the loaded cells from the nanowells to the microwells; loading a plurality of barcoded beads into the microwells so that a single bead occupies each cell-loaded microwell; capturing RNA from the cells and retrieving the RNA-loaded beads; and, sequencing the captured RNA.

In some embodiments, the microwell array comprises wells having a 50 μm diameter. In some embodiments, the nanowells have a diameter selected from: 10 μm, 15 μm and 20 μm. In some embodiments, the RNA is sequenced using one or more techniques comprising PCR. In some embodiments, the cells are loaded into the nanowells by applying a first alternating electrical potential.

In some embodiments, the method further includes loading a plurality of a second cell type into the nanowells. In some embodiments, the second cell type is loaded with a second alternating electrical potential.

In some embodiments, the method further includes inverting the aligned arrays so that the microwell array is beneath the nanowell array.

In certain aspects, the present invention relates to a DEP-trapping-nanowell-transfer (dTNT) system comprising: a single cell trapping nanowell array, and a microwell array pre-aligned on top of the nanowell array, wherein the microwell array is aligned with a microaligner device.

In some embodiments, the single-cell trapping nanowell array comprises wells having a dimeter selected from: 10 μm, 15 μm and 20 μm.

In some embodiments, the microaligner device is adapted and configured to align the wells of the nanowell array with the wells of the microwell array.

### BRIEF DESCRIPTION OF THE DRAWINGS

For a fuller understanding of the nature and desired objects of the present invention, reference is made to the following detailed description taken in conjunction with the accompanying drawing figures wherein like reference characters denote corresponding parts throughout the several views.

FIG. 1 depicts an exemplary design of a (DEP)-Trapping-Nanowell-Transfer (dTNT) sequencing (dTNT-seq) as contemplated herein. It shows a schematic illustration of the DEP-based single-cell mRNA sequencing platform and workflow. Two separate layers are pre-aligned and assembled using a custom-designed manipulator. After cell loading, single cells are actively trapped into nanowells by positive DEP. Subsequently, whole device is flipped and cells are transferred into larger microwells by gravity, followed by barcoded bead loading, cell lysis, and mRNA capture by the DNA oligomers on the surface of the beads, each containing a cell barcode and a unique molecular identifier (UMI). The beads are collected and pooled and the mRNAs are reverse transcribed in bulk, forming single-cell transcriptomes attached to microparticles (STAMPs). Amplification and tagmentation are then conducted for preparation of sequencing library. The sequencing data for transcriptome alignment is performed to generate gene expression matrix for downstream data analysis.

FIGS. 2A-2E depict an exemplary design and configuration of the dTNT device as contemplated herein. FIG. 2A depicts a top view of the DEP nanowell array for single cell trapping. FIG. 2B depicts an exemplary microscope image of the fabricated electroactive DEP array. FIG. 2C depicts an exemplary 3D optical surface profiler image of the cell capture nanowells. FIG. 2D depicts a cross sectional view of the assembled dTNT device, including the larger microwell layer on top, the DEP array chip at the bottom, and the PDMS gasket in between to form a flow channel for loading of cells, beads and all the reagents. FIG. 2E depicts a photo of a pre-aligned two-layer dTNT device assembled using a home-built aligner. Inset on the right: optical image showing the enlarged view of a representative region of the dTNT device.

FIGS. 3A-3E illustrate the evaluation of DEP-based single-cell trapping, occupancy rate, transfer efficiency, and bead loading. FIG. 3A depicts statistical analysis of cell numbers in 3080 nanowells in 35 regions imaged by fluoresce. In total, over 90% of the nanowells are occupied by single cells, and the doublet rate is less than 2%. FIG. 3B depicts a fluorescence image of single cells (green) trapped using the 10 μm depth nanowells. FIG. 3C depicts cell capture performance as a function of the nanowell depth. In the current study, the effect of 5, 10, 15 and 20 μm depth was investigated. The 10 μm nanowells resulted in the best single-cell trapping with a neglectable doublet rate. FIG. 3D depicts a fluorescence image of single cells transferred into large microwells. After flipping the dTNT device, an average of 82% trapped cells are successfully transferred. FIG. 3E depicts barcoded beads loaded to the microwells at a nearly 100% single bead occupancy rate due to the microwell size exclusion and the ability to move beads back-and-forth in the flow channel.

FIGS. 4A-4E depicts single-cell RNA sequencing of species-mixing samples using dTNT-seq. FIG. 4A depicts a fluorescence image of mouse 3T3 (green) and human HEK (red) cells on the DEP nanowell array. FIG. 4B depicts sequencing reads mapped to human vs mouse genomes. (FIGS. 4C and 3D) Violin plots showing # of genes or transcripts detected in single cells (center-line: median; limits: first and third quartile; whiskers, ±1.5 IQR; points; values, >1.5 IQR) FIG. 4E depicts a comparison of human gene capture efficiency with that in Seq-Well using a PBMC sample.

FIGS. 5A-5D depicts graph-based unsupervised clustering analysis and the comparison with non-electrode method (scFTD-seq). FIG. 5A depicts UMAP visualization of two major species-specific groups generated using dTNT, each of which has three distinct single-cell clusters. FIG. 5B depicts heatmap expression of top 5 differentially expressed gene markers in each cluster in dTNT-seq. FIG. 5C depicts UMAP visualization of single cell transcriptomes generated using scFTD-seq. Same as that in dTNT, two major species-specific groups were identified and each of which has two larger subpopulations. FIG. 5D depicts the cell number of each major cluster in dTNT-seq and scFTD-seq.

FIGS. 6A-6D depicts a comparison of biological processes underlying the identified clusters between dTNT-seq and scFTD-seq through GSEA analysis. Top 10 GO terms enriched in the cluster (FIG. 6A) DEP_Human 0; (FIG. 6B) Human 0; (FIG. 6C) DEP_Mouse 1; (FIG. 6D) Mouse 1. The representative GSEA enrichment plot and the distribution of marker gene that define each cluster were also showed. GO terms were ranked by the normalized enrichment score (NES) generated from GSEA.

FIG. 7 depicts a complete fluorescence image after the trapped single cells transfer. By turning the device upside down, an average of 82% trapped single cells are successfully transferred into the underneath larger microwells.

FIG. 8 depicts a complete fluorescence image of barcoded beads loading for mRNA capture. Barcoded beads loading rate can be nearly 100% due to the microwell size exclusion and the ability to move beads back-and-forth.

FIG. 9 depicts an exemplary workflow of dTNT-seq operation and the processing time of each step.

FIG. 10 depicts a large-area fluorescence image of species-mixed human and mouse cells trapping by DEP. Here, mouse (3T3) cells are labeled with green fluorescent dye and human (HEK) cells are labeled with red fluorescent dye.

FIG. 11A-11C depict an assessment of the single cell resolution and transcriptome quality. FIG. 11A demonstrates that more than 90% of the transcripts align to the species-specific genome for majority of cells. FIG. 11B illustrates the relationships between the number of transcripts and the percentage of mitochondrial genes. FIG. 11C illustrates the relationships between the number of transcripts and the number of genes. Cells are filtered based on gene counts (between 200 to 5000) and the percentage of mitochondrial genes (less than 10%) to exclude low-quality cells or potential cell doublets.

FIGS. 12A-12D illustrate the identification of highly variable features and linear dimensional reduction (PCA). FIG. 12A depicts 2000 genes that exhibit high cell-to-cell variation in the dataset (i.e., they are highly expressed in some cells, and lowly expressed in others) are selected for the downstream analysis and the top 10 most highly variable genes are labeled. FIG. 12B depicts visualizing cells that define the PCA. FIG. 12 C a JackStraw plot. In the JackStraw plot, there is a sharp drop-off in significance after the first 7 PCs. FIG. 12D shows that in the Elbow plot, one can observe an ‘elbow’ around PC8-9. It is advised to err on the higher side when choosing this parameter according to the Seurat protocol, so we choose 10 PCs as inputs to perform UMAP clustering.

FIGS. 13A and 13B depict exemplary heatmaps of the top 15 enriched genes found to define each cluster for (FIG. 13A) human species and (FIG. 13B) mouse species.

FIGS. 14A-14C depict the top 10 GO terms enriched in the cluster (FIG. 14A) DEP_Human 1; (FIG. 14B) Human 1; (FIG. 14C) DEP_Human 2. The GSEA enrichment plots of top 3 GO terms were also showed. GO terms were ranked by the normalized enrichment score (NES) generated from GSEA.

FIGS. 15A-15C depicts the Top 10 GO terms enriched in the cluster (FIG. 15A) DEP_Mouse 0; (FIG. 15B) Mouse 0; (FIG. 15C) DEP_Mouse 2. The GSEA enrichment plots of top 3 GO terms were also showed. GO terms were ranked by the normalized enrichment score (NES) generated from GSEA.

FIG. 16 diagrams the design pattern and workflow of pairing two types of cells for studying cell-to-cell interactions through scRNA-seq.

FIGS. 17A and 17B demonstrate an exemplary “Roof DEP nanowell array” embodiment of the invention where the nanowell array directly captures single cells on top without requiring inverting. FIG. 17A depicts a schematic illustration of this method. FIG. 17B depicts a cross-sectional illustration of top-loading the nanowell array with cells.

FIG. 18 depicts an exemplary embodiment where an addressable “Roof DEP nanowell array” device was designed to enable flexible manipulation of cells of interest.

### DEFINITIONS

The instant invention is most clearly understood with reference to the following definitions.

As used herein, the singular form “a,” “an,” and “the” include plural references unless the context clearly dictates otherwise.

Unless specifically stated or obvious from context, as used herein, the term “about” is understood as within a range of normal tolerance in the art, for example within 2 standard deviations of the mean. “About” can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about.

As used in the specification and claims, the terms “comprises,” “comprising,” “containing,” “having,” and the like can have the meaning ascribed to them in U.S. patent law and can mean “includes,” “including,” and the like.

Unless specifically stated or obvious from context, the term “or,” as used herein, is understood to be inclusive.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 (as well as fractions thereof unless the context clearly dictates otherwise).

### DETAILED DESCRIPTION OF THE INVENTION

The present invention provide an integrated dielectrophoresis (DEP)-Trapping-Nanowell-Transfer (dTNT) system and methods for high throughput single-cell RNA sequencing (scRNA-seq). Specifically, the present invention provides an integrated dielectrophoresis (DEP)-Trapping-Nanowell-Transfer (dTNT) approach, referred to as a dTNT-seq, to perform cell trapping and bead loading both in a sub-Poisson manner to facilitate scRNA-seq.

dTNT-seq System

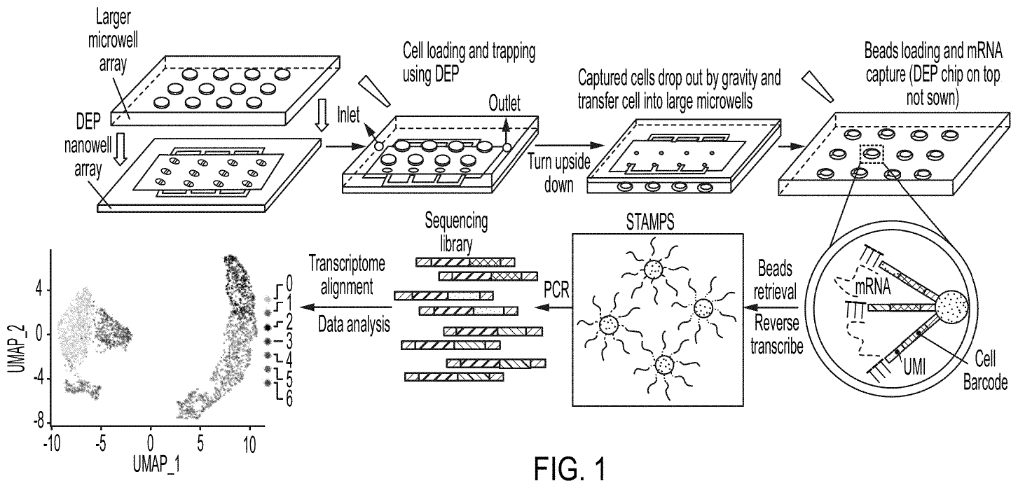

Referring now to FIG. 1, the dTNT-seq system of the present invention includes a DEP-based single-cell mRNA sequencing platform. The system includes a microwell array slide, a DEP nanowell array slide, and a micro-aligner for precisely aligning the wells of the nanowell array slide and the microwell array slide when assembled.

The microwell array slide includes an array of wells sized to accommodate DNA barcode beads used in scRNA-seq analysis. Each of the microwells in the array can be sized to have a diameter of about 50 In some embodiments the microwells have a diameter in the range of from about 20 μm to about 30 from about 30 μm to about 40 from about 40 μm to about 50 from about 50 μm to about 60 from about 60 μm to about 70 from about 70 μm to about 80 and any and all increments therebetween. In certain embodiments, each of the microwells in the array have a depth of about 50 The microwells can have a depth of from about 20 μm to about 40 from about 40 μm to about 60 from about 60 μm to about 80 and any and all increments therebetween. Embodiments of the microwells have a pitch of about 100 The microwells can have a pitch of from about 60 μm to about 80 from about 80 μm to about 100 from about 100 μm to about 120 and any and all increments therebetween.

The microwell array slide may be fabricated from any suitable material as understood in the art. For example, embodiments of the microwell are fabricated from a polymethylmethacrylate (PMMA). In some embodiments, the microwells are fabricated directly in a layer of SU-8 coated on the PMMA substrate. In some embodiments, silicon or glass are used as the substrate materials for fabricating SU-8 microwells.

The microwells are positioned along a series of microfluidics channels positioned across the substrate. The substrate may also include fluid access holes through silicon or glass for introducing beads.

The nanowell array slide includes a DEP trap. Embodiments of the nanowell array slide include an array of nanowells sized to isolate single cells. Each of the wells can have a diameter of about 20 μm. Embodiments of the nanowells have a diameter of up to about 5 μm, from about 5 μm to about 10 μm, from about 10 μm to about 15 μm, from about 15 μm to about 20 μm, from about 20 μm to about 25 μm, and any and all increments therebetween. In some embodiments, the diameter is 5 μm, 10 μm, 15 μm, or 20 μm. The nanowells have a depth of about 20 μm. Embodiments of the nanowells can have a depth of from about 5 μm to about 10 μm, from about 10 μm to about 15 μm, from about 15 μm to about 20 μm, rom about 20 μm to about 25 μm, and any and all increments therebetween. The nanowells have a pitch matched with the pitch of the microwells on the microwell array slide. For example, the nanowells can have a pitch of about 100 μm. The nanowells are aligned along microchannels formed in the slide

The microwell array slide and the DEP nanowell array slides can be precisely aligned and assembled. In some embodiments, a gasket is positioned between the two slides when assembled in order to form a flow channel for loading cells, beads, reagents, and the like. The gasket may have a thickness of about 100 The thickness may be from about 50 μm to about 75 from about 75 μm to about 100 from about 100 μm to about 125 from about 125 μm to about 150 and any and all increments therebetween. The gasket may be constructed from any suitable material as understood in the art including for example PDMS.

Each of the nanowell array slide and microwell array slide may include a plurality of wells. For example, the array slides may have up to 2000 wells, from about 2000 wells to about 2200 wells, from about 2200 wells to about 2400 wells, from about 2400 wells to about 2500 wells, from about 2600 wells to about 2800 wells, from about 2800 wells to about 3000 wells, from about 3000 wells to about 3200 wells, from about 3200 wells to about 3400 wells, from about 3400 wells to about 3600 wells, from about 3600 wells to about 3800 wells, from about 3800 wells to about 4000 wells, from about 4000 wells to about 4200 wells, from about 4200 wells to about 4400 wells, from about 4400 wells to about 4600 wells, from about 4600 wells to about 4800 wells, from about 4800 wells to about 5000 wells, and any and all increments therebetween.

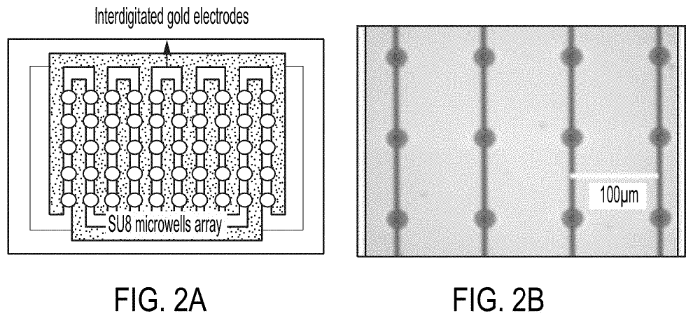

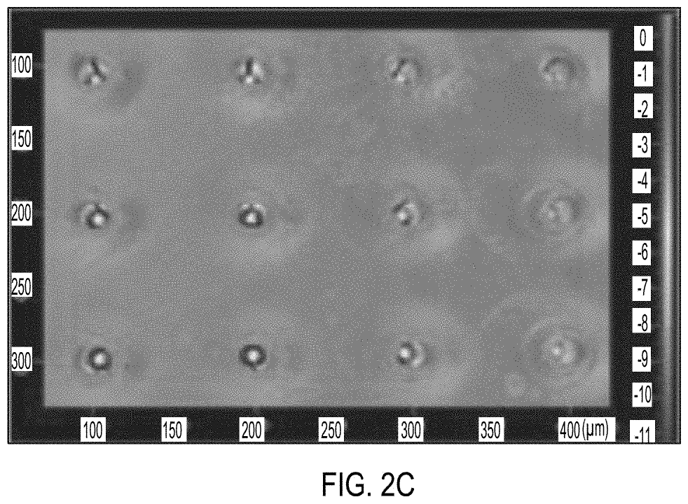

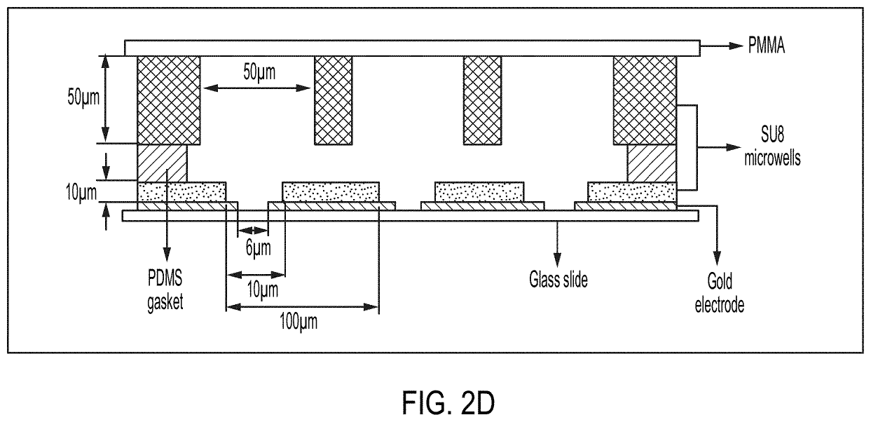

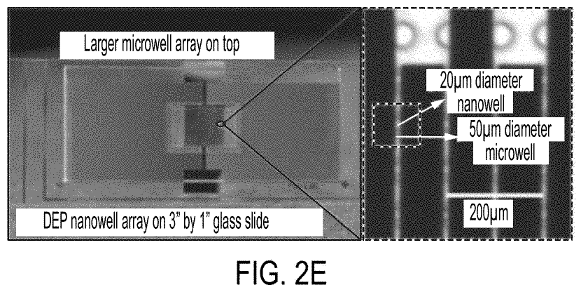

The microwell array slide and nanowell array slide can be assembled so that the wells in each slide are perfectly aligned. In certain embodiments, the two slides are aligned using the aligner shown in FIG. 2E. The aligner as contemplated herein ensures the alignment of the nanowells and microwells which ensures efficient cell trapping and transfer. The m

In certain embodiments, the system of the present invention is assembled so that the nanowell array slide is on bottom of the microwell array. In this case, the assembled system is inverted once cells are loaded into the DEP nanowell array slide. The microwell array slide and nanowell array slide can also be aligned by one or more pins, slots, bosses, or other mechanical devices. The microwell array slide and nanowell array slide can also be aligned using imaging (e.g., optical imaging) before being fixed to each other (e.g., with adhesive).

In some embodiments, the system of the present invention is assembled so that the nanowell array slide is on the top of the microwell array slide. In this case, the cells are loaded into the nanowell array while in the top “roof” position or orientation.

### Methods

The present invention provides methods for high-throughput single cell sequencing using the dTNT device as contemplated herein.

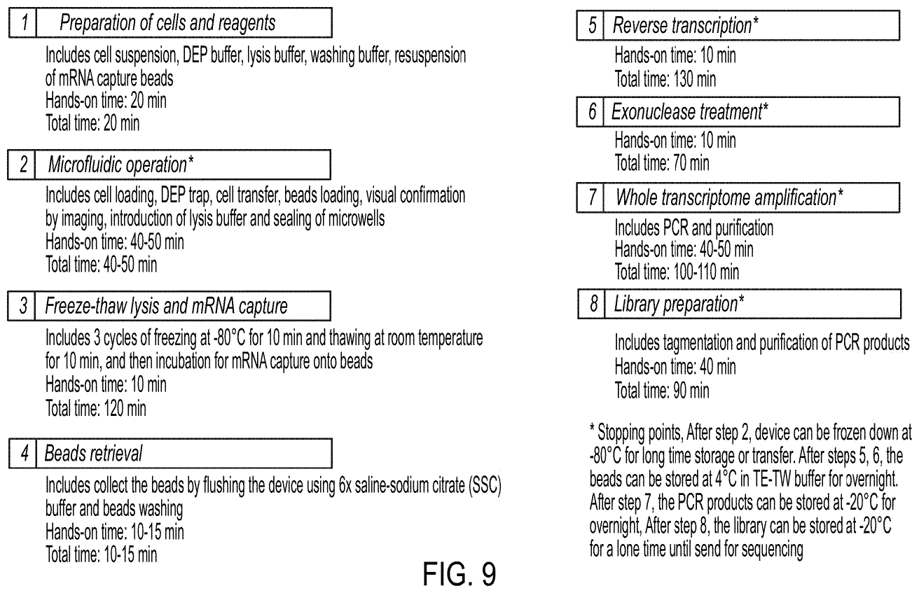

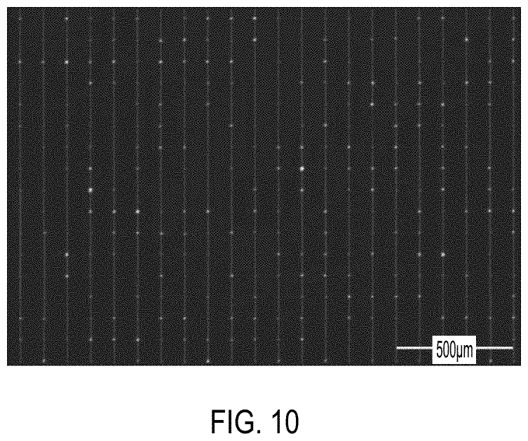

Referring now to FIG. 1 and FIG. 9, embodiments of the methods are outlined in the schematic and workflow diagrams the dTNT-seq of the present invention

Embodiments of the methods include first aligning the microwell array precisely on top of the DEP nanowell array. The wells of each of the array slides are aligned using a micro-aligner device. In some embodiments, a gasket is positioned between the microwell array and the nanowell array in order to form channels between the two slides.

Embodiments of the methods include loading cells are loaded into the microfluidics channels positioned along the nanowell slide. The cells may include any cell type as understood in the art, as preferred for use. For example, the cells may include primary cells, immortalized cells, stem cells, and the like. The cells may include cells isolated from any suitable species as understood in the art, including, for example, murine, human, rattus, rabbit, bovine, porcine, canine, equine, and the like. The cells may include cells isolated from one or more suitable tissues including for example, vascular tissue, blood, muscle tissue, nerve tissue, bone tissue, breast tissue, prostate tissue, heart tissue, pancreas tissue, and the like, including normal tissue an cancer tissue and/or cells. The cells may include one or more cells types including mouse embryonic fibroblast cells (NIH 3T3), human embryonic kidney (HEK293) cells, human peripheral blood mononuclear cells (PBMCs), including human monocytic-like cells (U937), lung cancer cells (NCIH1975), prostate cancer cells (DU145 and PC3), breast cancer cells (MCF-7) and HeLa cells, and one or more combinations thereof.

Embodiments of the methods include applying a voltage to the DEP nanowell array. The applied voltage may include an applied alternating electrical potential. Embodiments of the applied alternating electrical potential can include a peak-to-peak (Vp-p) potential of 4 V, where V is the peak to peak voltage. The electrical potential may include up to 4 V, about 4 V to about 6 V, from about 6 V to about 8 V, from about 8 V to about 10 V, from about 10 V to about 12 V and any and all increments therebetween. Embodiments of the electrical potential is applied in a sinusoidal electrical wave at a frequency of about 10 MHz. The frequency can be in the range of from about 0.1 MHz to about 1 MHz, from about 1 MHz to about 10 MHz, from about 10 MHz to about 100 MHz, and any and all increments therebetween. The voltage is applied to the DEP chip for cell trapping via a positive DEP effect. The applied voltage is applied in order to trap a quanta of cells in each of the nanowells. The quanta of cells can be equal to a quanta of electrode pairs in each of the nanowells. In some embodiments, the quanta of cells is 1.

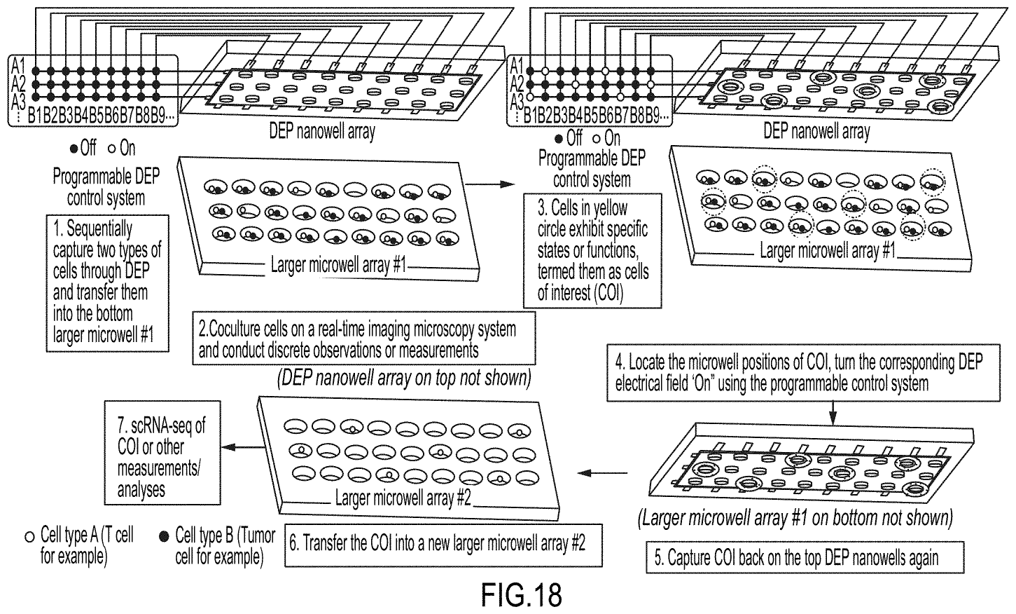

In some embodiments, as depicted in FIG. 18, a second voltage can be applied to the DEP nanowell array (e.g., to a second pair of electrodes). In some embodiments, a second cell type is loaded with the second applied voltage (e.g., after trapping of a first cell type and flushing of remaining cells of the first cell type). In such embodiments, the quanta of cells is 2. In certain application of the present invention, the quanta of cells if more than 2.

Embodiments of the methods include inverting the aligned arrays so that the microwell array is beneath the nanowell array. That is, when single-cell DEP trapping is completed, the device is turned upside down. Electricity can continue to be applied to the DEP nanowell array during inversion in order to hold the trapped cells against gravity. When the electricity is discontinued, the trapped cells will be pulled by gravity into the aligned microwell below the nanowell.

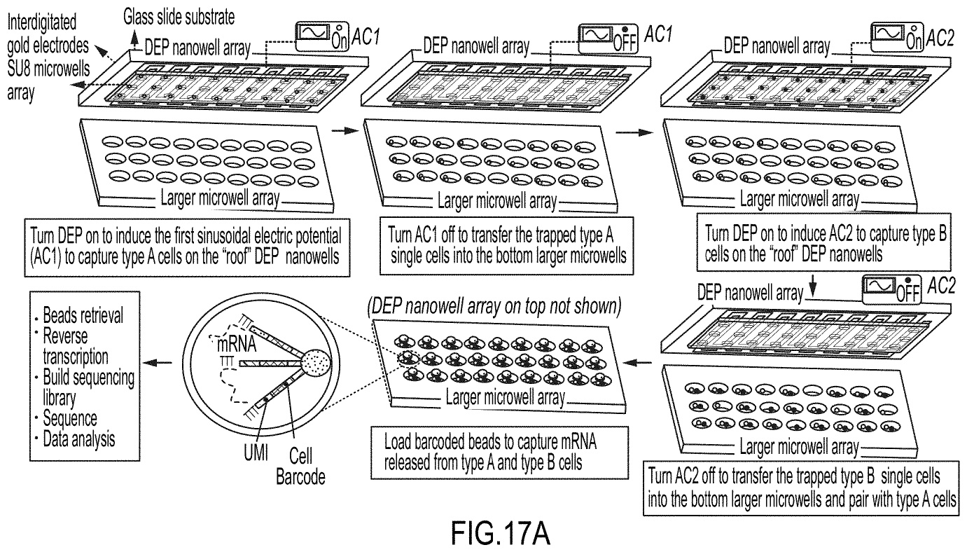

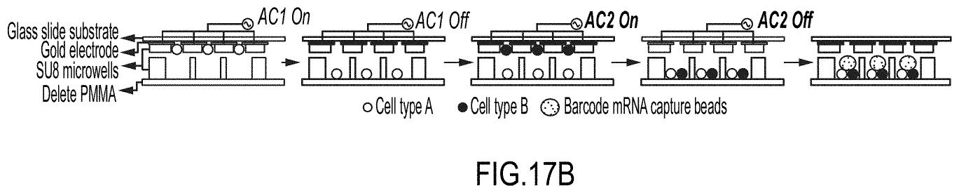

In some embodiments, as depicted in FIGS. 17A and 17B, cells are loaded into the nanowell array slide with the nanowell array positioned on top of the microwell array. That is, the nanowell array is oriented when cells are loaded. In such embodiments, the inverting step is not necessary.

Embodiments of the methods include discontinuing applying electricity to the nanowell array. That is, when the DEP trapping voltage is turned off, the loaded cells are transferred from the nanowells to the aligned microwells. In some embodiments, the loaded cells are transferred by gravity. However, positive pressure, vacuum, and/or vibration could also be utilized to promote movement from the nanowells to the microwells.

Embodiments of the methods include loading a plurality of barcoded beads into the microwells so that a single bead occupies each cell-loaded microwell.

Embodiments of the methods include capturing RNA from the cells and retrieving the RNA-loaded beads. That is, barcoded beads, lysis buffer and fluorinated oil are sequentially loaded to produce an array of sealed cell-bead pairs. The cells are lysed using one or more techniques including for example a freeze-thaw lysis method in order to release mRNAs for single-cell transcriptome sequencing.

Embodiments of the methods include sequencing the captured RNA. That is, the mRNAs captured onto barcoded beads by the DNA oligomers on the surface of the beads, each containing a cell barcode and a unique molecular identifier (UMI). The captured mRNAs are reverse transcribed in bulk to form single-cell transcriptomes attached to microparticles (STAMPs). The captured RNA is amplified using suitable techniques as understood in the art including for example PCR amplification of cDNAs synthesized from the RNA, In some embodiments, purification, and sequencing library preparation. IN some embodiments, sequencing data for transcriptome alignment is performed to generate gene expression matrix for downstream data analysis. In certain aspects, this aspect of the invention allows for decoupling of cell trapping and bead loading so that each can be done in a sub-Poisson manner and the cells can recirculate back-and-forth on the DEP trap array until reaching a capture rate of greater than 90%.

### EXPERIMENTAL EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

### Example 1

### Background

High-throughput single-cell RNA sequencing (scRNA-seq) analysis has emerged as a powerful tool for cell identification and classification in a variety of research fields including immune responses, tumor microenvironments and cancer heterogeneity. Utilizing droplet-based encapsulation or subnanoliter well array to pair single cells and uniquely barcoded mRNA capture beads, it is feasible to profile gene expression of tens of thousands of single cells in a massively parallel manner and at a relatively low cost. For instance, a total of 14,813 lung cells were mapped to 30 cell types to quantify changes in cellular activity states across young and old mice, the transcriptional landscape of 17,374 mouse bone marrow vascular, perivascular and osteoblast cell populations were obtained to show previously unappreciated levels of cellular heterogeneity within the bone marrow niche, and an atlas comprising 47,016 CD45+ cells were collected from 8 primary breast carcinomas unraveling phenotypic expansion of intratumoral immune cells. The improvement of microfluidics techniques, combined with the maturation of clustering algorithms and statistical modeling for scRNA-seq, have transformed the capability to dissect complex biological systems at the single-cell level. In nearly all high-throughput scRNA-seq methods, molecular barcoding using uniquely barcoded beads is the enabling and indispensable step, which allows us to use a bulk RNA-seq workflow while maintaining single-cell resolution.

While a range of microfluidic-based platforms have been developed to improve reproducibility, throughput and sensitivity, there are some fundamental limitations. For droplet-based approaches, cell suspension, oligonucleotides barcoded beads and reagents are co-flowed into the system, followed by encapsulation in nanoliter droplets. The incessant workflow allows for efficient single cell isolation, however, also induces low cell-bead pairing efficiency, which impedes its adoption for low input samples. Further, the unavoidable requirement of peripheral equipment limits their portability for remote clinical centers. Alternatively, micro or nanowell-based devices provide a simplified strategy that is less costly, more portable, and potentially compatible with highly limited patient samples. By specifically adjusting the well dimensions, single cells and barcoded beads can be co-isolated into subnanoliter wells with over 95% bead loading efficiency. However, in order to minimize the rate of two or multiple cells in the same well, called cell duplets or multiplets, the cells need to be loaded in a super-Poisson manner such that only less than 10% of bead-occupied wells receive cells.

In recent years, many techniques have been developed to improve the performance of single-cell capture and beads loading. For example, to capture as many cells as possible from a limited sample, Zheng et al. devised a droplet-based system using the GemCode beads, which enabled 3′ mRNA digital counting of tens of thousands of single cells at approximately 50% of cells capture efficiency. To avoid the multiple-beads-in-a-drop resulted from random encapsulation, Moon et al. utilized a spiral channel-based microfluidics platform to order the highly concentrated beads with equal spacing before co-encapsulating cells, which generated a deterministic encapsulation of beads and diminished barcoding errors. To minimize the beads loss during the Drop-seq implementation, Biočanin and colleagues re-designed the original dropleting device to make it compatible with both air-pressure systems and syringe pumps and devised an accompanying chip for highly efficient retrieval of the mRNA capture beads. However, despite these technical improvements, all the aforementioned methods still follow a stochastic passive approach to pair single cells and mRNA capture beads. Very recently, Cheng et al. developed Hydro-Seq, which allows for contamination-free scRNA-seq of circulating tumor cells (CTCs) at a high cell capture efficiency (72.85±2.64%). However, the size-based isolation configuration only functions when target cells, such as CTCs, are typically larger than other unwanted cells (blood cells), limiting its use in a broad range of scRNA-seq applications.

Therefore, there is still an unmet need for developing a high throughput approach to capture thousands of single cells in the sub-Possion regime for scRNA-seq. Dielectrophoresis (DEP) trapping is a well-studied active trapping mechanism and can be a potential solution to address this challenge. Since Pohl and Hawk pioneered the separation of live and dead yeast cells using DEP in 1966, this label-free approach has been used for manipulating and sorting bacterial and mammalian cells. Recent studies include a 3D electrodes-based cell separation device that can remove 99.1% of RBCs in a blood sample spiked with 1% cancer cells at a processing rate of ˜170,000 cells per second, an electroactive double well array that can analyze intracellular materials at the single-cell level with minimal target cell loss, and a planar chip for high-throughput cell-cell pairing at up to 74.2% pairing efficiency. In addition, several commercialized products incorporating DEP for cell manipulation have been used for determining particle size, isolating tumor cells and detecting biomarkers.

However, the extension of the success in single-cell DEP trapping to nanowell-based scRNA-seq is not trivial, comprising major obstacles including the inherent incompatibility of optimal trapping conditions between large-sized DNA-barcoded beads and the cells of interest that are much smaller. In order to retain sufficient surface area and barcoded oligomers to capture the whole transcriptome of single cells, the DNA-barcoded mRNA capture beads are typically ˜40 μm in diameter and if possible, should be even larger. However, the diameter of the DEP trap wells has to be less than 20 μm for efficient and selective single cell trapping with low probability of capturing doublets. Moreover, the depth of DEP trap nanowells need to be less than 15 μm because the cell capture efficiency decreases sharply with increasing the well depth. Therefore, it is not feasible to directly combine the trapping of barcoded beads and single cells in the same well for single-cell RNA sequencing.

In the present study, dTNT-seq is reported, which is a highly integrated DEP-Trapping-Nanowell-Transfer (dTNT) device for active trapping of single cells in a small 20 μm-wide nanowells and transfer to a larger-sized 50 μm wells for the loading of barcoded beads to perform single-cell mRNA transcriptome capture. The device consists of a roof chip and a bottom chip to separately implement the capture of cells and the loading of beads, respectively, both in the sub-Poisson regime. As shown schematically in FIG. 1, a 50 μm-microwell array slide was first pre-aligned on top of the 20 μm DEP nanowell array using a custom-designed micro-aligner. When single-cell DEP trapping is completed, the device is turned upside down so that the trapped cells can be transferred to the underneath larger microwells after switching off the DEP trapping voltage. Then, barcoded beads, lysis buffer and fluorinated oil were sequentially loaded to produce an array of sealed cell-bead pairs and a freeze-thaw lysis method was employed to release mRNAs for single-cell transcriptome sequencing (see the scFTD-seq protocol we previously reported). In brief, the mRNAs captured onto barcoded beads are reverse transcribed in bulk to form single-cell transcriptomes attached to microparticles (STAMPs), followed by a PCR amplification of the synthesized cDNAs, purification, and sequencing library preparation. Such a configuration allows us to decouple cell trapping and bead loading so that each can be done in a sub-Poisson manner and the cells can recirculate back-and-forth on the DEP trap array until reaching a capture rate >90%. Here, the inventors devised and fabricated an interdigitated dTNT device, which contained 3,600 arrayed electroactive DEP traps in single-cell capture nanowells that are integrated with the matched large microwell array. A single-cell trapping rate of ˜92% and a high transfer efficiency of 82% was demonstrated. Together with the bead loading rate >99%, we can break the Poisson limit for both cells and beads, in other words, in a double-sub-Poisson distribution. Finally, the performance for scRNA-seq was evaluated by profiling a mixture of mouse fibroblast NIH3T3 cells and human embryonic kidney HEK293 cells, which were quantitatively compared to scRNA-seq data obtained using the previously reported devices. The dTNT-seq is a successful demonstration of double-sub-Poisson scRNA-seq in high throughput (thousands of single cells per run) enabled by a DEP active cell trapping mechanism and attributed in part to the non-trivial engineering to integrate all steps in a fully packaged microdevice.

### Results and Discussion

Design of the dTNT-seq Device

The fundamental principles of DEP trapping are described in Methods. The dTNT device is composed of an electroactive DEP nanowell array chip for single cell capture and a larger microwell chip for accepting transferred cells and loading DNA-barcoded beads. To generate non-uniform electric field for cell trapping, interdigitated gold electrodes with a 6-μm gap were patterned on the glass substrate. The entire surface was then coated with an SU8 insulating layer (˜10 μm in thickness), in which 3,600 nanowells (60 by 60) were fabricated to expose the DEP trap electrodes (FIG. 2A, 2B). The nanowells are smaller than 20 μm in diameter in order to achieve single cell capture with negligible doublets. To confirm the quality of microfabricated DEP trap nanowells, a 3D optical surface profiler (Nexview™ NX2, Zygo) was used to perform non-contact measurement of the SU8 nanowells, and the result shows nanowell structures are fully developed and the dimensions match the original design (FIG. 2C).

The size of DEP nanowells dedicated for single-cell capture is not sufficient for accommodation of DNA barcode beads used in scRNA-seq. To resolve this issue, we devised an array of larger microwells on a polymethylmethacrylate (PMMA, also known as Plexiglas or Acrylic) substrate. These larger microwells are ˜50 μm in diameter and depth and ˜100 μm in pitch, which shared the same pitch size with the DEP trap array chip. Instead of using PDMS to fabricate microwells, the 50 μm microwells are directly fabricated in a layer of SU8 coated on the PMMA substrate to avoid the shrinkage problem (PDMS shrinks ˜1.5% after curing) that may result in difficulty in alignment with the DEP array chip. Silicon or glass can be used as the substrate materials for fabricating SU-8 microwells although it requires special techniques to drill fluid access holes through silicon or glass. Consequently, to easily fabricate the inlet holes and connect a tubing for introducing cells and beads, PMMA is chosen as the substrate material for the fabrication of large SU-8 microwells. Two parts—a DEP nanowell array chip and a 50 μm microwell array chip—are precisely aligned and assembled (FIG. 2D). A 100 μm-thickness PDMS gasket was placed in between to form a flow channel for controlled loading of cells, beads, and reagent perfusion. Using our home-built aligner/manipulator, a perfect vertical alignment of the two separated layers can be realized to ensure efficient cell trapping and transfer (FIG. 2E).

Single Cell Trapping with DEP

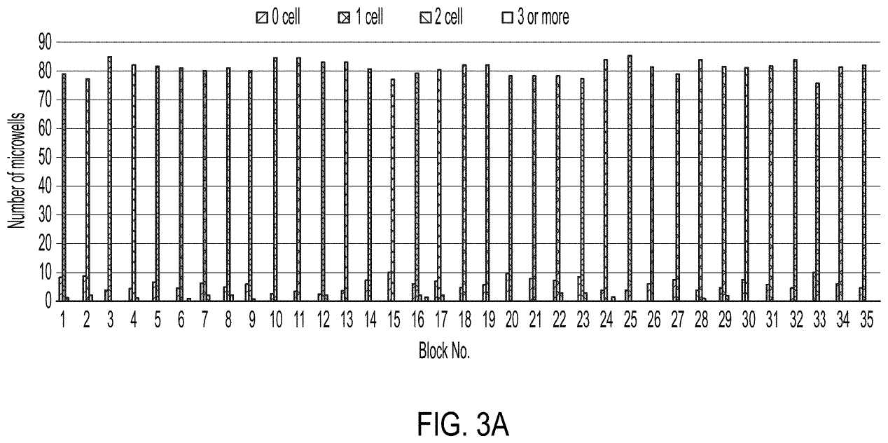

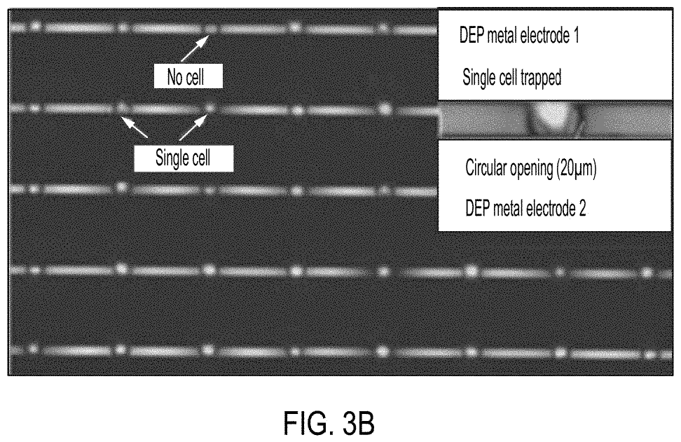

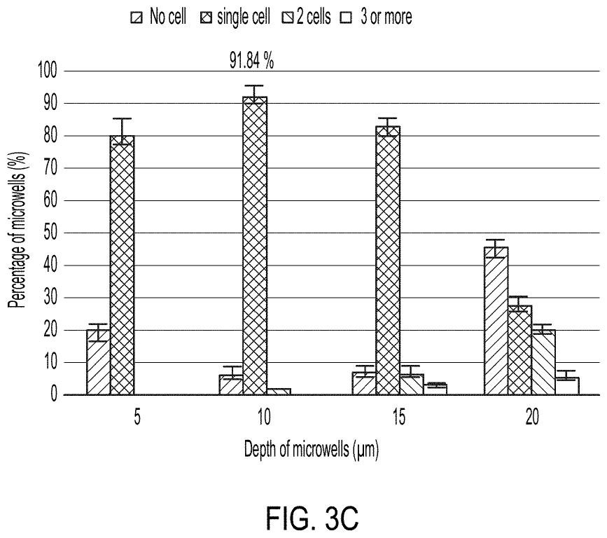

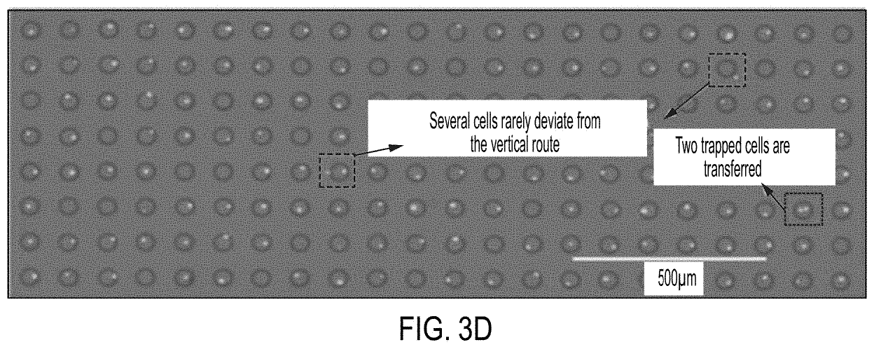

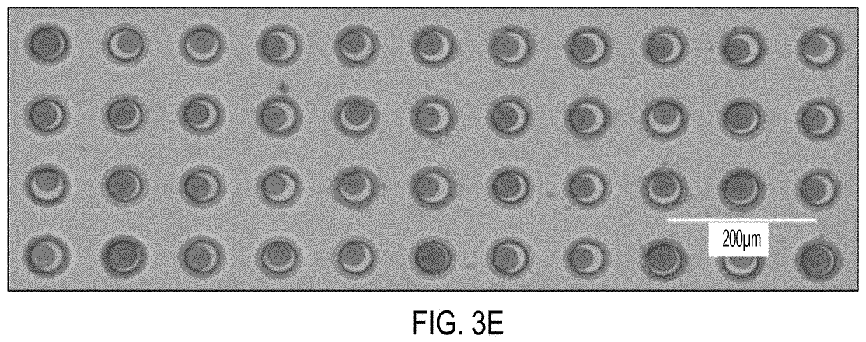

Mouse embryonic fibroblast cells (NIH 3T3) were used to validate the overall dTNT-seq workflow and its technical performance. After device priming with DEP buffer, the green fluorescently-stained cells (20 μL, 40,000 cells) were loaded through an inlet port and delivered at a flow rate of 1 μL/min to fill the entire microchannel. While the cell suspension flowing through the whole nanowell array, a 4V peak-to-peak (Vp-p) sinusoidal electrical wave at 10 MHz was applied to the DEP chip for cell trapping via a positive DEP effect. From the real time imaging recorded by an EVOS FL Auto Microscope (Life Technologies/Thermo Fisher), it was observed that the DEP force is adequate to pull cells down from the flow stream toward the nanowells when they were approaching the nanowell edges. Almost all nanowells were gradually occupied by single cells after 5 min. For low input samples (<5,000 cells), this step can be done by recirculating the cells in the microfluidic chamber until all single cells are trapped. Then, the flow rate was increased to 20 μL/min to flush out excessive cells while keeping DEP on. We examined 35 representative regions, each containing 88 nanowells, yielding a total of 3,080 nanowells to score the number of cells in each compartment (FIG. 3A). One of the trapping images is shown in FIG. 3B. In a set of 3 independent experiments using nanowells of 10 μm in depth, our device demonstrated an average of 91.84% of single-cell trapping rate with less than 2% doublets, outperforming any existing high-throughput scRNA-seq platforms in terms of single-cell occupation ratio. Cell viability is >95% as confirmed by the trypan blue exclusion assay after DEP trapping.

To evaluate the effect of nanowell depth on single cell trapping, DEP nanowells were fabricated with various depths of 5, 10, 15 and 20 μm, and conducted cell capture experiments independently. The amplitude and frequency of the applied sinusoidal AC driving voltage and the cell suspension flow rate remained the same as the aforementioned parameters. A 5-15 μm depth configuration was found to provide a satisfactory single-cell capture rate, while the probability of capturing more than one cell in a single nanowell increases with the depth (FIG. 3c). For 20 μm depth configuration, on average, over 26% of the nanowells trap two or more cells, which is not acceptable for the downstream scRNA-seq analysis. Even if the applied electric potential is increased to 10V (Vp-p), we observed that ˜40% of nanowells are still vacant after 10 min of DEP trapping. The result indicates that the DEP force decreases dramatically with the increase of nanowell depth. For 5 μm-deep nanowells, almost no doublets were observed because the localized DEP effect excludes the accommodation of two or more cells at this dimension. However, during excessive cell removal at a flow rate of 20 μL/min, some captured single cells escape from the nanowells, thereby decreasing the overall single-cell trapping rate. Lowering the flow rate can solve this problem but may lead to increased microfluidic trapping time and reduced cell viability. However, this remains to be systematically tested. According to these results, the 10 μm nanowells provide an optimal condition for single cell trapping with a neglectable doublet rate.

Cell Transfer and Bead Loading with dTNT

A key feature of the dTNT device is the use of a two-layer design to separately perform cell capture and bead loading. After the DEP cell trapping, the whole device was flipped (cells remain trapped on the “ceiling” when the DEP voltage is still on) to transfer single cells into the larger microwells used for loading beads afterwards. Once the whole device is slipped, the electric potential is switched off. The device is left on the bench undisturbed for 10 min, which is sufficient to allow most of the trapped cells to exit DEP nanowells and fall into the 50 μm microwells by gravity. Utilizing this capture-and-transfer tactic, although a small number of the trapped cells are still stuck to the DEP chip after waiting for 10 min, the dTNT device still demonstrated a high transfer efficiency with the average transfer rate of 82% (FIG. 3d and Figure S1 for complete image). Taken together for a 3,600 microwell device at a single-cell capture rate of 91.84%, we transferred 2,700 single cells into the large microwells for scRNA-seq. The area of the DEP trap array in our current device is small. It can be readily scaled up to the trapping of >10,000 single cells per run.

The rationale to design this modular dTNT device is that the dimensions of nanowells used for single-cell DEP trapping are too small to accommodate the barcoded beads (˜50 μm) currently used widely for scRNA-seq. Cell transfer into larger microwells provides enough room for bead loading, subsequent co-isolation with single cells, and capture of single-cell-derived mRNA transcriptomes on beads. However, after turning the device upside down, the inlet is now on the bottom of the dTNT device. To solve this problem, instead of pipetting the barcoded beads suspension on the inlet port, it is first aspirated into a tubing connected with syringe and then perfused into the channel manually. Finally, excessive beads are washed out and the oil is loaded to prevent cross contamination during cell lysis. With the microwell size exclusion and the ability to move beads back-and-forth within the flow chamber, we routinely achieved >99% single bead loading efficiency (FIG. 3E and FIG for complete image).

This dTNT device outperforms other established scRNA-seq methods in terms of the overall cell-bead pairing efficiency (Table 1). Specifically, in Drop-Seq, only ˜5% of bead-encapsulated droplets contain single cells; for InDrop method, the narrow constraint design to squeeze and slow down the passage of hydrogel beads increases the pairing rate to >10%; for other nanowell based approaches like SeqWell, the loaded beads are expected to cover tens of thousands nanowells to get several thousand cell-bead pairs, so most of the beads are not utilized to capture single-cell-derived mRNAs, resulting a significant waste of expensive DNA-barcode beads. By contrast, for our dTNT device, although we still use microwells to co-isolate beads and cells, the active cell capture mechanism allows us to break the Poisson limit at the initial cell trapping step and achieved >2,700 single-cell data points from as few as 3,600 bead-located microwells, which is significantly less than those required by other microwell methods such as SeqWell. With a ˜75% cell-bead co-isolation rate, a notable advantage of our device is that it reduces bead consumption by effectively making the best use of all microwells, reducing the cost substantially because the barcode beads are the most expensive reagent in current scRNA-seq workflow. What is more, in spite of the fact that dTNT is a relatively complex integrated device, it does not necessarily increase the processing time in comparison to the established approaches. In FIG. 9, the operation time of each step is listed and the stopping points are indicated. After beads loading and microwell sealing, the device can be stored at −80° C. for a long time, which allows cell/beads capture at distributed sites such as small clinics or point-of-care settings but the downstream library preparation and sequencing done after shipping to a centralized facility.

TABLE 1
Comparison of the cell-bead pairing efficiency across
different high-throughput scRNA-Seq platforms.

| | | | Cell/bead pairing |
|---|---|---|---|
| | Cell occupancy | Beads capture | rate (Droplets/ |
| | rate (Droplets/ | rate (Droplets/ | wells co-occupied |
| | wells containing | wells containing | with single cells |
| Method | single cells %) | single beads %) | and beads %) |
| Drop-Seq | ~5-10% | ~5-10% | <5% |
| InDrop | ~10% | ~80-100% | ~10-50% |
| 10× | ~10% | ~80-100% | ~10% |
| Chromium | | | |
| SeqWell | 5%-10% | ~95% | ~5%-10% |
| dTNT-seq | ~75% | ~99% | ~75% |

### Single-Cell RNA Profiling of Species Mixed Samples

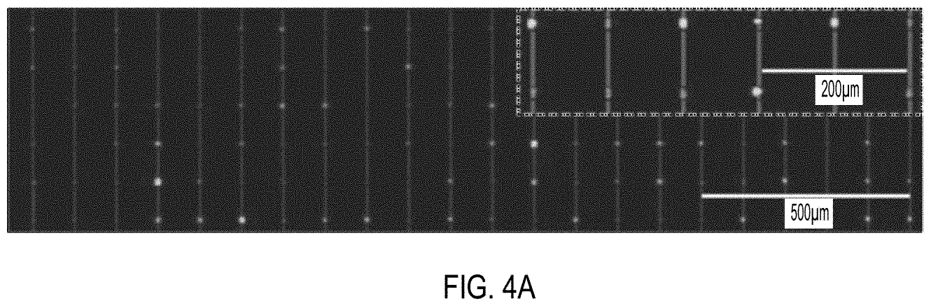

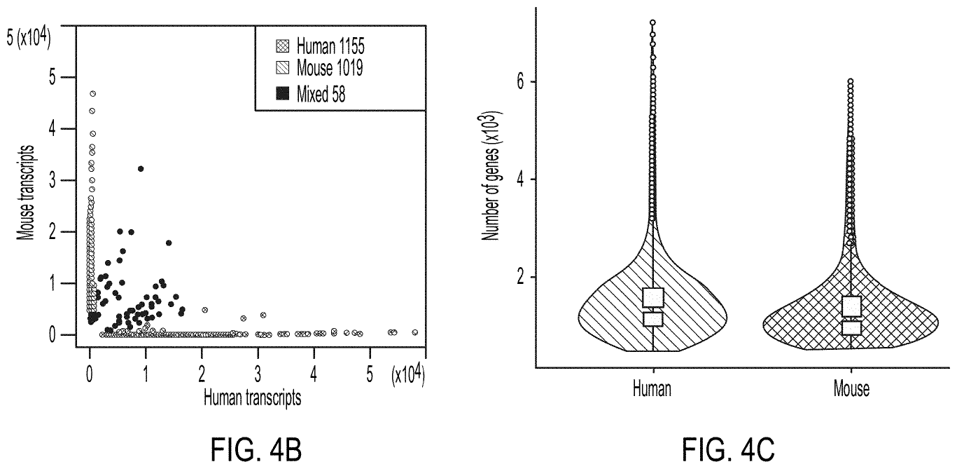

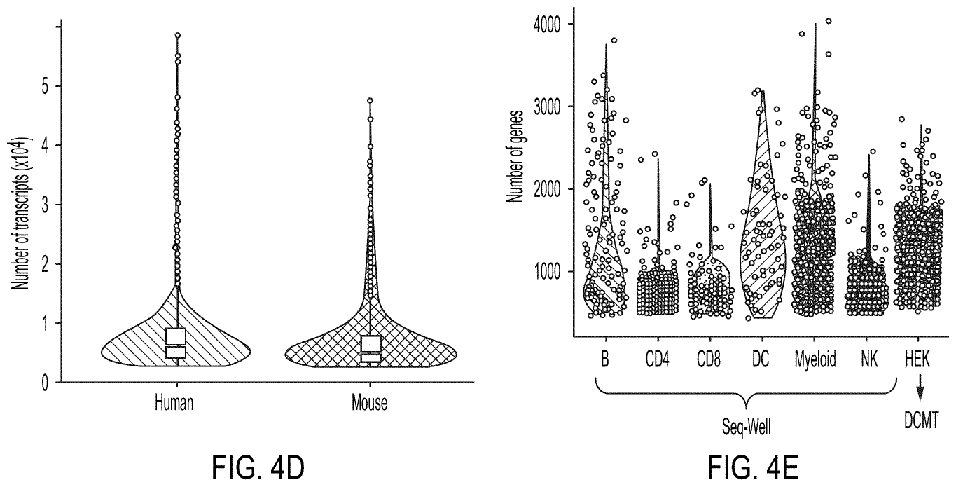

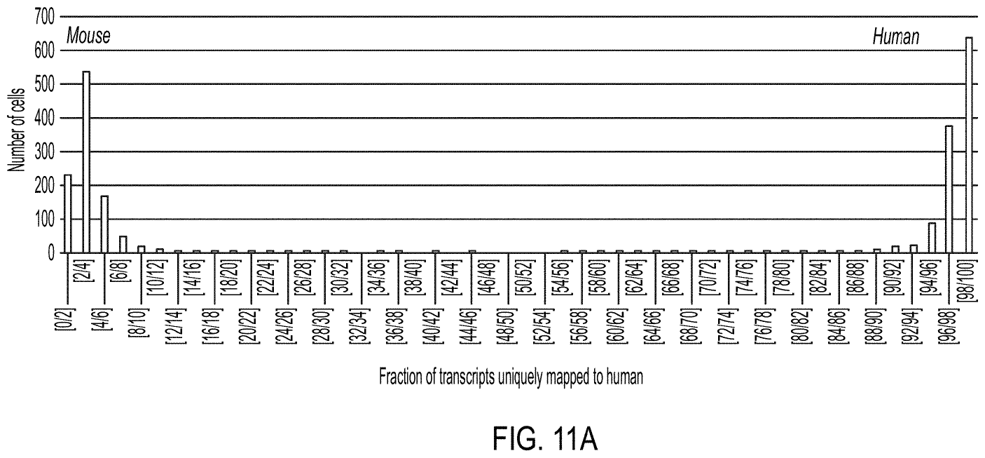

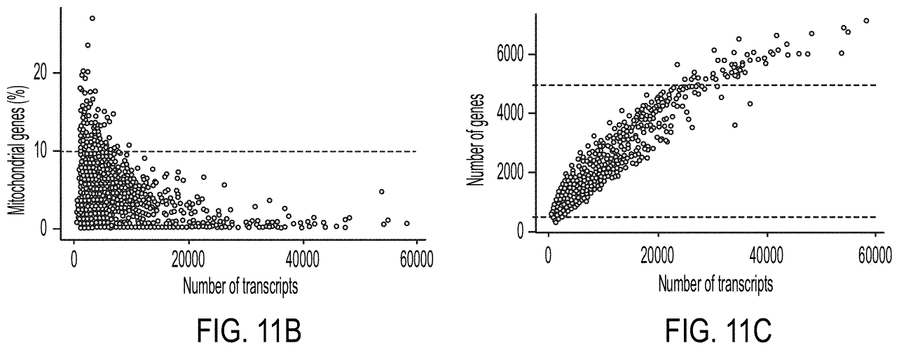

To evaluate the performance of dTNT-seq for single-cell transcriptome sequencing, we profiled a 1:1 mixture of human embryonic kidney HEK293 cells and mouse embryonic NIH3T3 fibroblast cells. For the characterization of DEP capture efficiency, HEK cells with red fluorescent dye and 3T3 cells with green fluorescent dye were loaded into the device (FIG. 4a and Figure S4 for complete image). After bead loading, mRNA capture, reverse transcription, amplification, library preparation and sequencing at a nominal depth of ˜30,000 reads/cell, we aligned the reads to the mixed human:mouse reference genomes, yielding 2,748 single-cell transcriptomes. After a strict filtering process, we still obtained 2,232 high-quality single-cell transcriptomes and this data was used for the species distinction analysis. In total, we obtained the transcriptomic profiles of 1,155 HEK cells and 1,019 3T3 cells, which was close to the expected 1:1 mixing ratio. Most transcripts align either to mouse or human genome for most of the cells and only 2.6% (58 cells) of the identified cells had a mixed phenotype, suggesting highly species-specific single-cell mRNA profiles and minimal cross-contamination (FIG. 4B and FIG. 11A). We detected a median of 5,367 mRNA transcripts from 1,225 genes in HEK cells and 4,459 mRNA transcripts from 1,012 genes in 3T3 cells (FIG. 4C, 4D).

Although DropSeq, 10×Genomics Chromium system and SeqWell all provided their transcript capture efficiency and accuracy using HEK:3T3 species mixture, they only sequenced several hundred cells at saturating depth (average 200 k reads/cell), which is 5-10 folds higher than the depth in our work. Here, to keep cell number and sequencing depth commensurate, we compare our results with the SeqWell data obtained from human peripheral blood mononuclear cells (PBMCs), which were found to be comparable to each other (FIG. 4E).

Comparison with Non-DEP Capture Methods for Performance in Clustering and GSEA

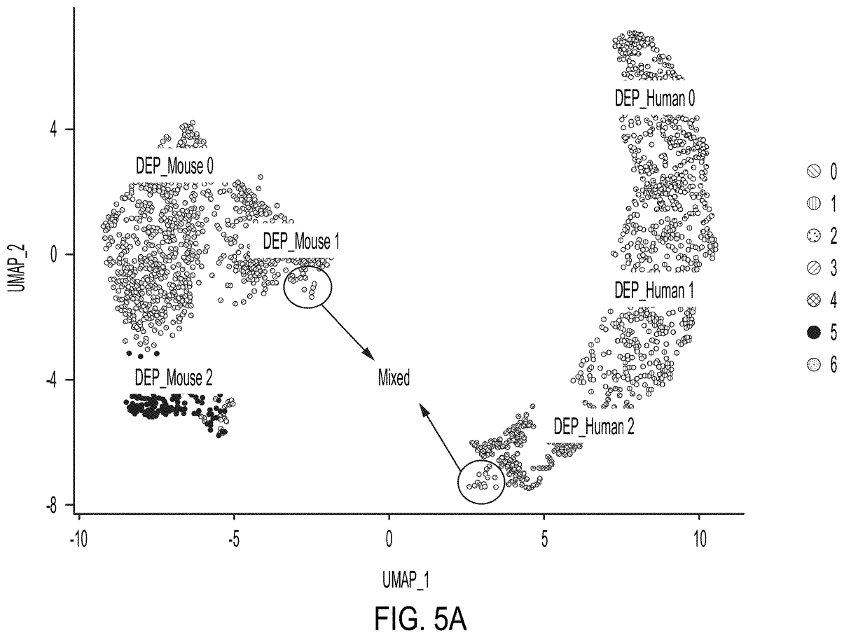

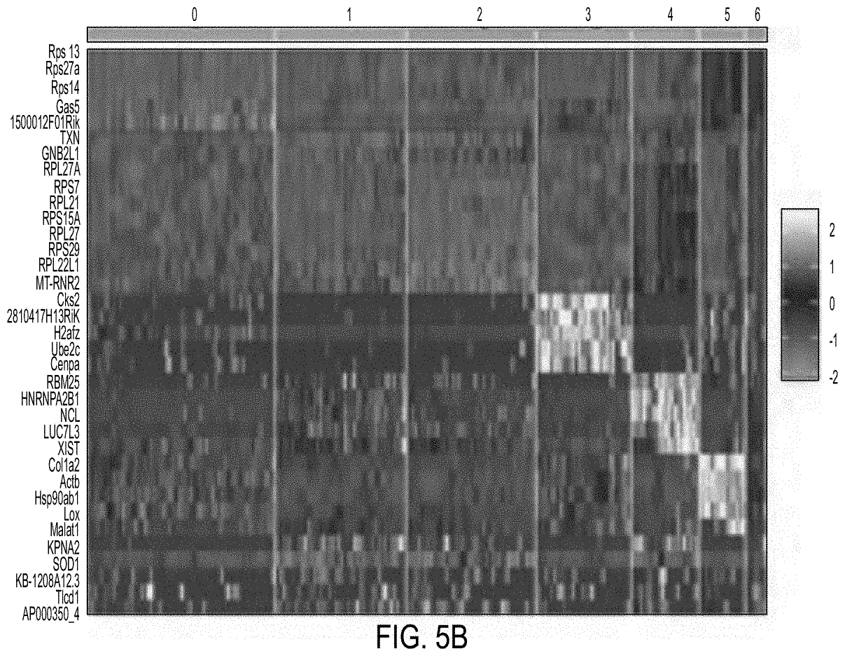

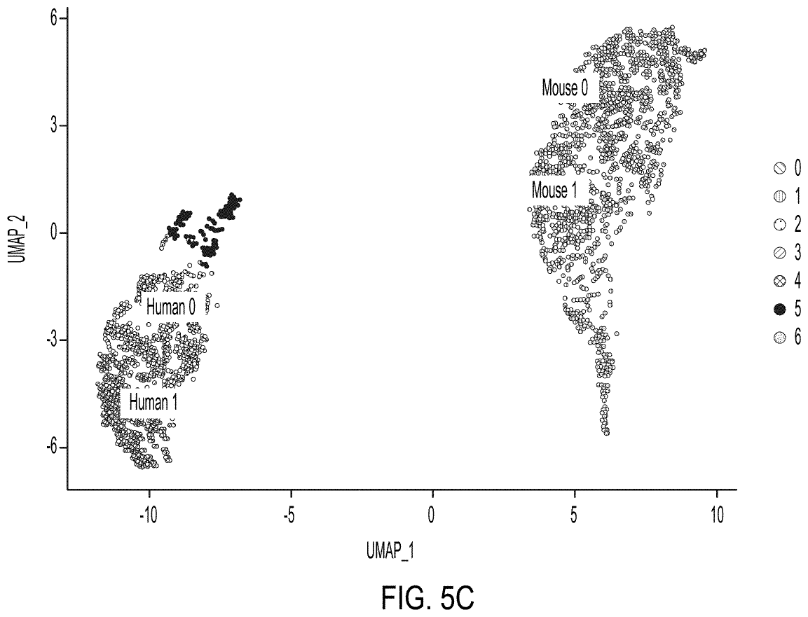

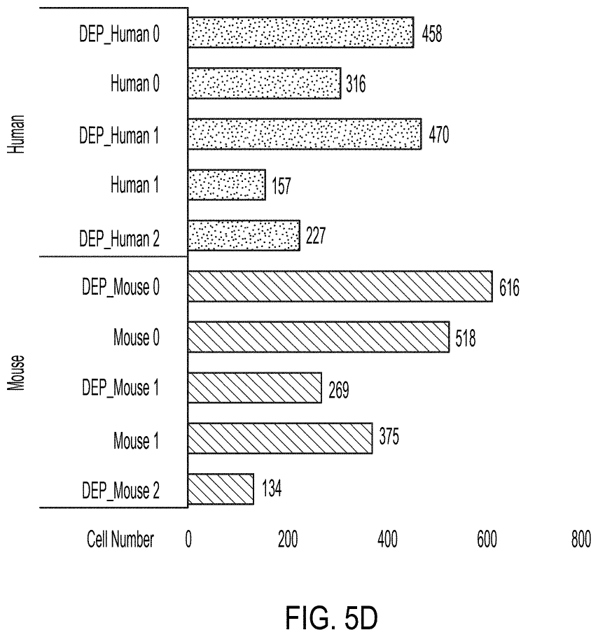

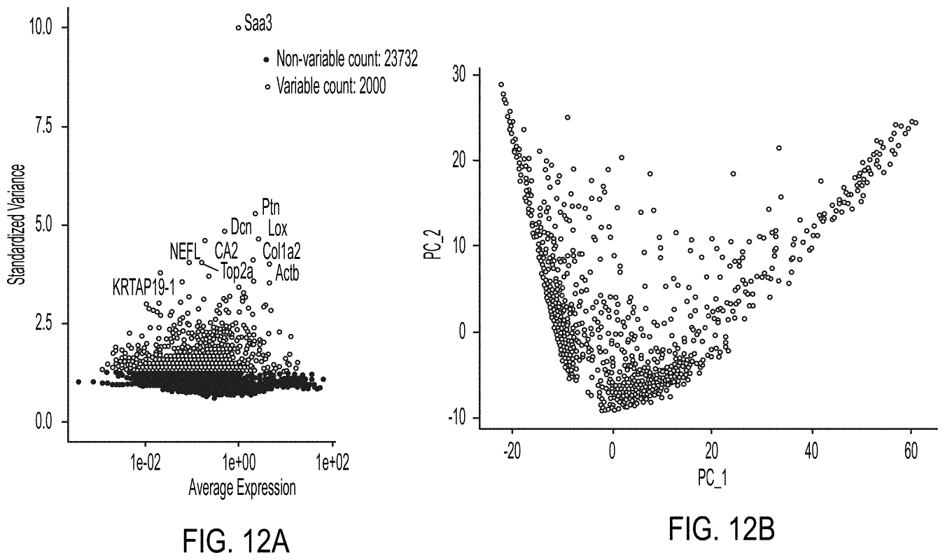

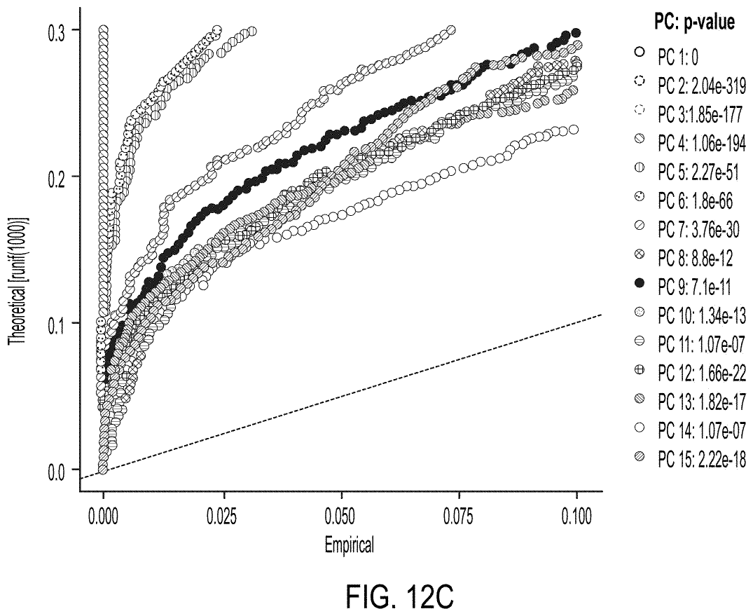

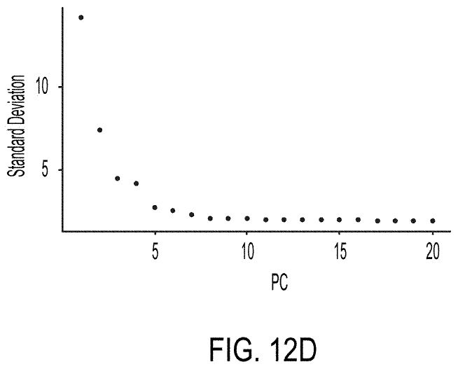

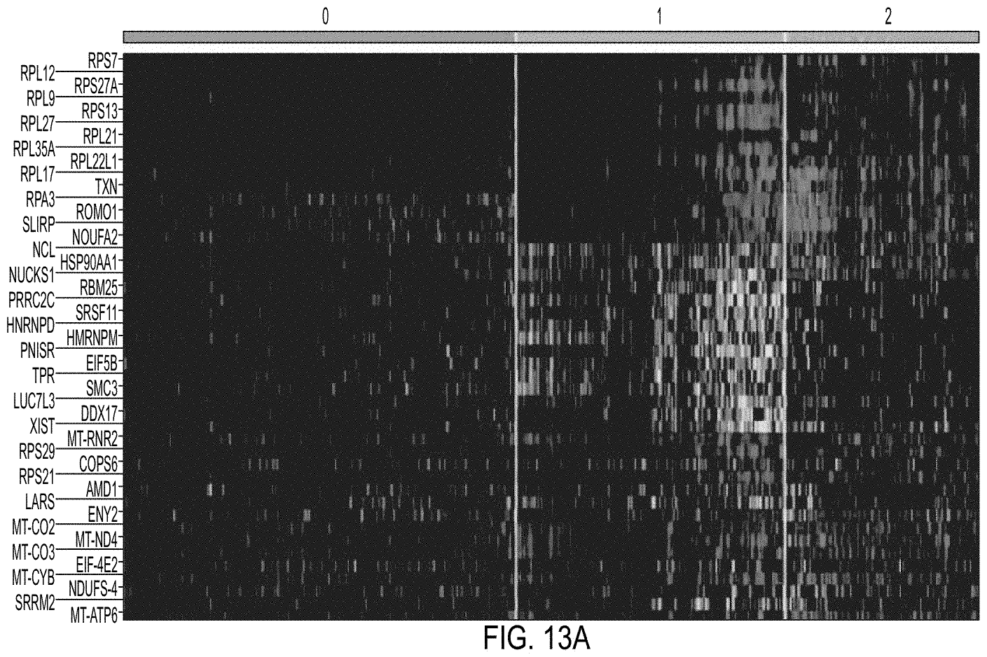

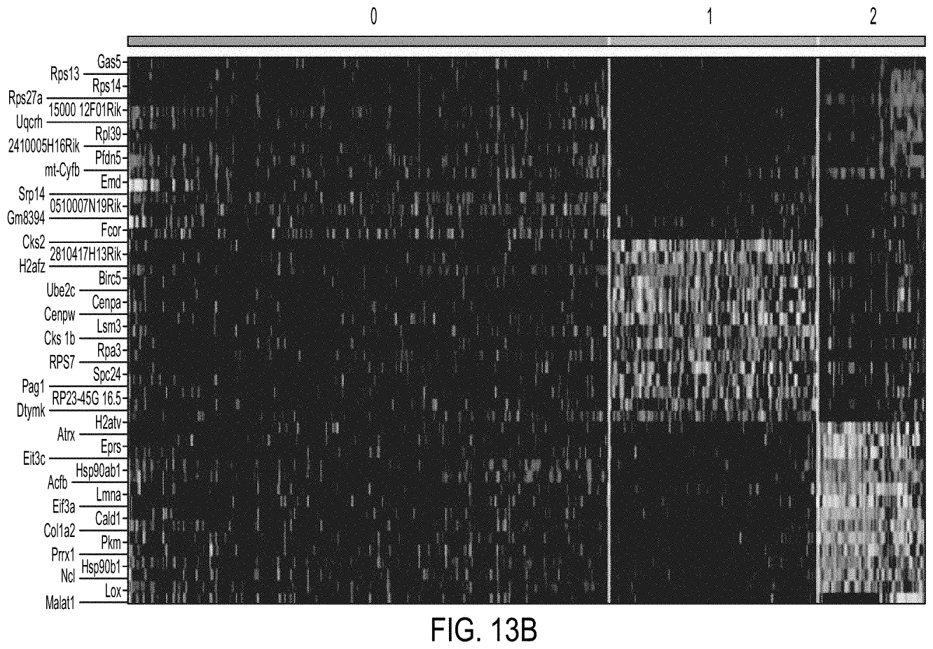

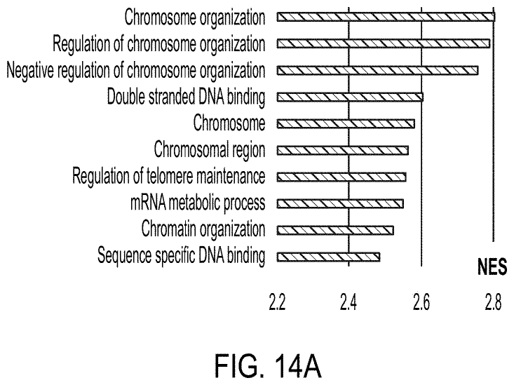

Unsupervised graph-based clustering algorithm was performed to analyze our sequencing data and the results are visualized in the Uniform Manifold Approximation and Projection (UMAP) graphs. Following the standard pre-processing workflow of quality control, data normalization and scaling, we selected a subset of 2,000 genes that exhibit high cell-to-cell variation in the dataset to perform linear dimensional reduction using Principal Component Analysis (FIGS. 12A and 12B). Based on the JackStraw plot and the Elbow plot, top 10 statistically significant PCs were employed as inputs to project single cells onto a two-dimensional map (FIGS. 12C and 12D). As shown in the UMAP visualization (FIG. 5a), cells are well separated into two major categories, each of which is identifiable as the corresponding cell type determined by the species-specific genes (FIG. 5B). We also generated a top 15 differentially expressed gene heatmap for three DEP_Human and DEP_Mouse sub-clusters, respectively (FIG. 13). A question that might arise is that our DEP capture approach exposes single cells to high electric field, which could potentially alter transcriptional profiles and scRNA-seq data. To address this concern, we compared dTNT-seq to the data generated using non-DEP trapping microwell-based methods reported previously to investigate whether the use of DEP affects single-cell transcriptional profiles. Using the non-DEP microwell-based method called scFTD-seq, single cells are stochastically loaded into the microwell arrays and then co-isolated with the DNA barcoded beads. Except for the cell capture and transfer step, both platforms use the same biochemistry workflow including a freeze-thaw lysis method to release mRNAs, reverse transcription, PCR amplification, and tagmentation to prepare sequencing library. The inventors performed the same 3T3:HEK species mixture experiment using scFTD-seq, and sequenced the library at the same depth. After filtering, 1595 single cell transcriptomes that consists of 956 mouse cells and 639 human cells were obtained. Subsequently, UMAP clustering analysis was performed and the result showed the major groups similar as those obtained by dTNT-seq (FIG. 5C). To inquire different gene regulatory pathways and biological processes underlying the observed clusters, we profiled 4 larger subpopulations (Human 0 and 1, Mouse 0 and 1) in scFTD-seq and 6 subpopulations (DEP_Human 0, 1, 2 and DEP_Mouse 0, 1, 2) in dTNT-seq using gene set enrichment analysis (GSEA) and drew a comparison between DEP-based and non-DEP based approaches (FIG. 5D).

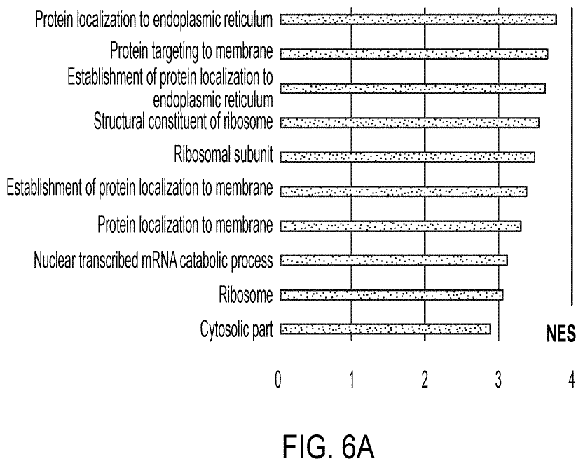

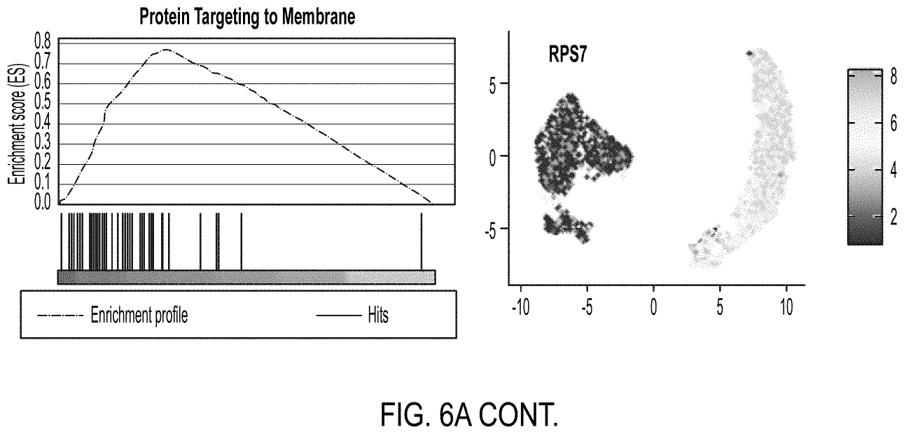

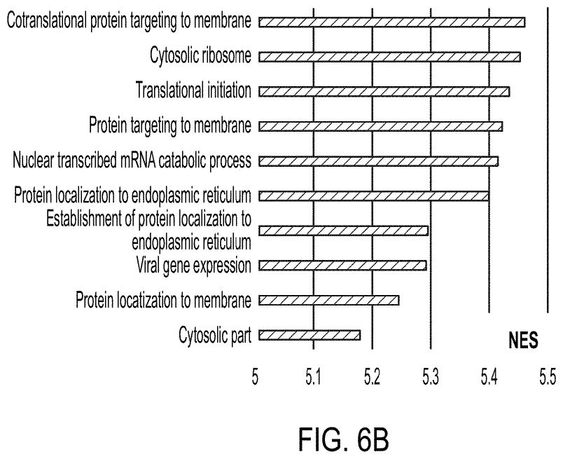

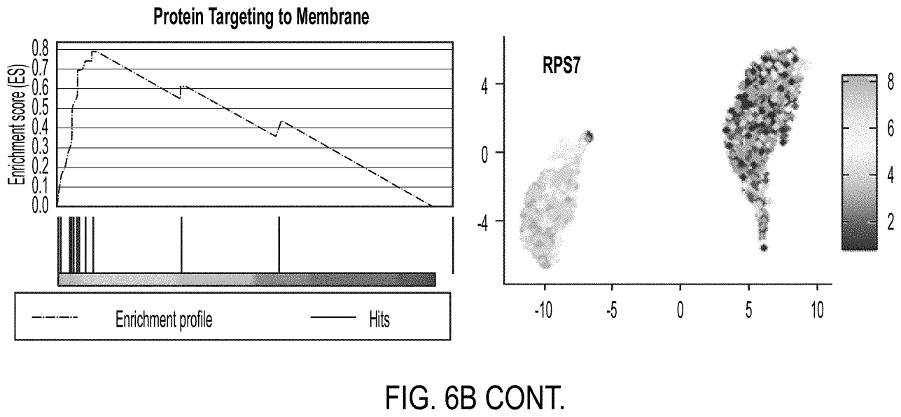

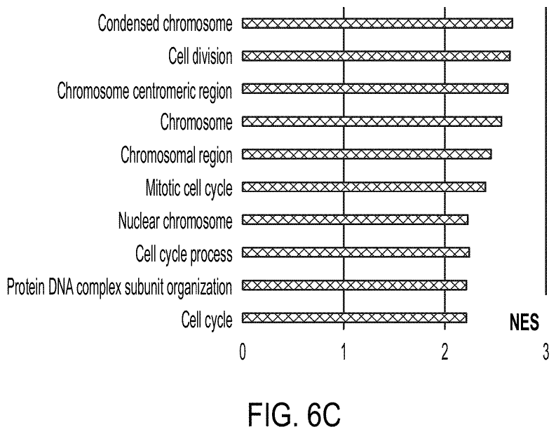

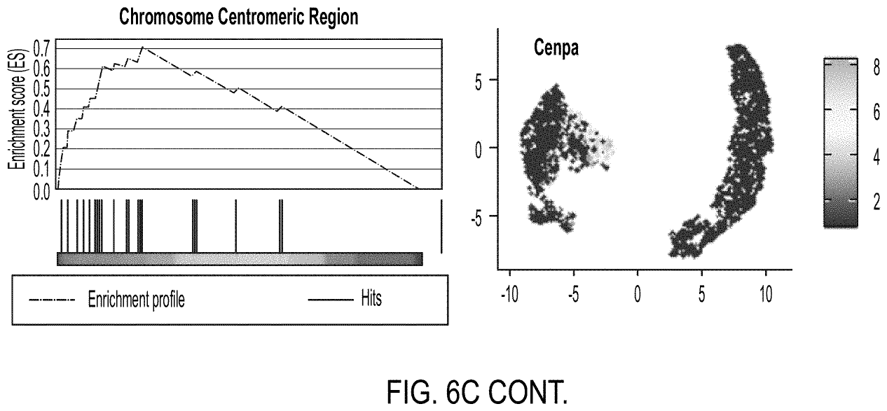

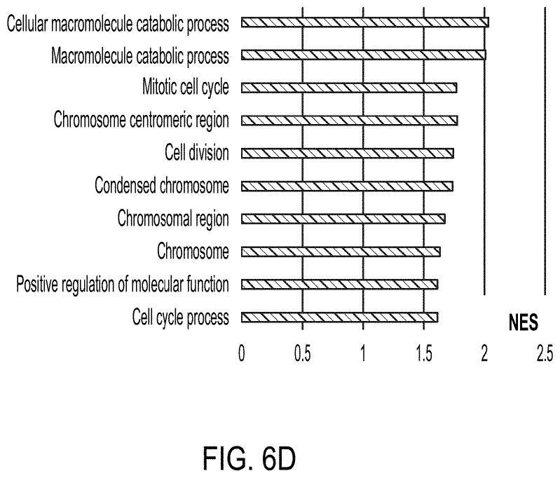

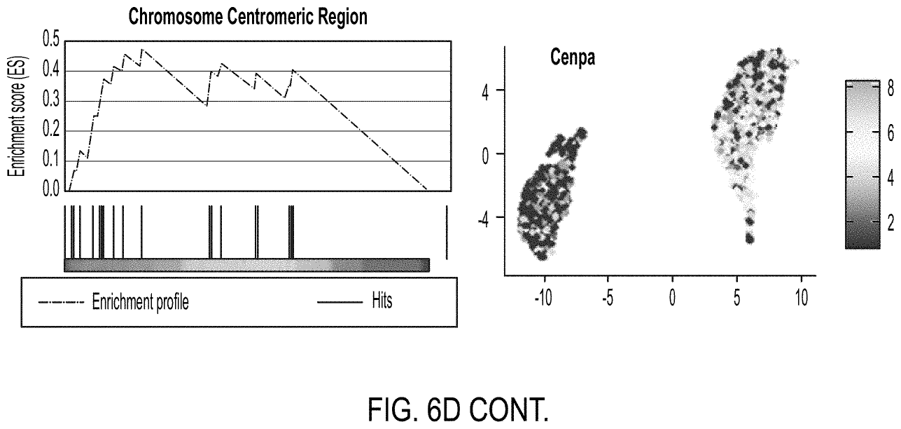

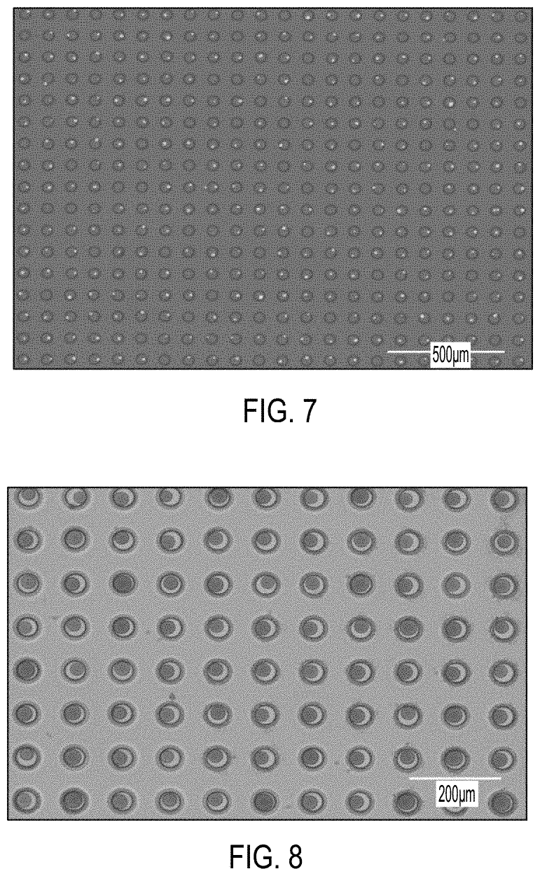

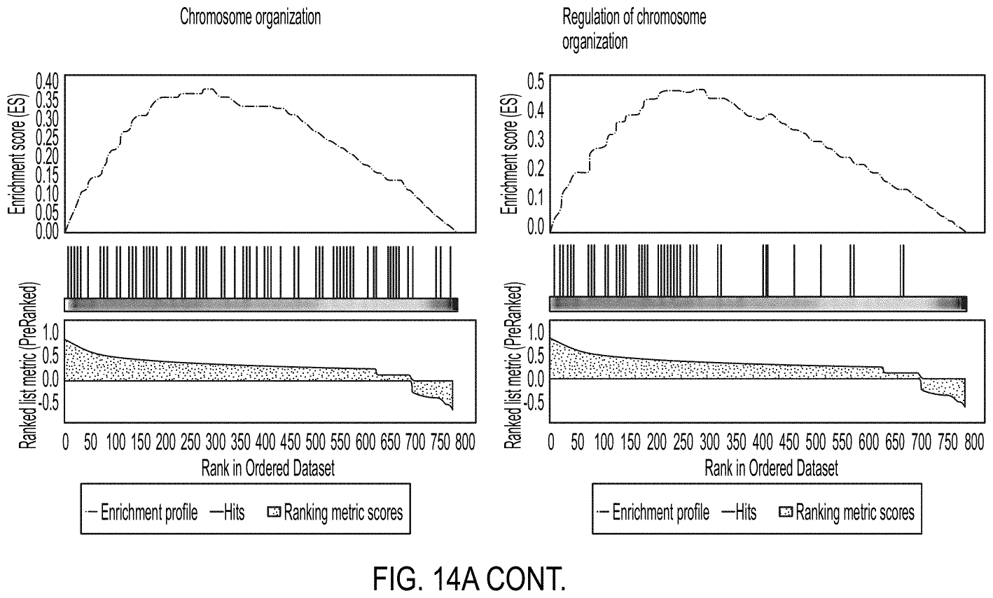

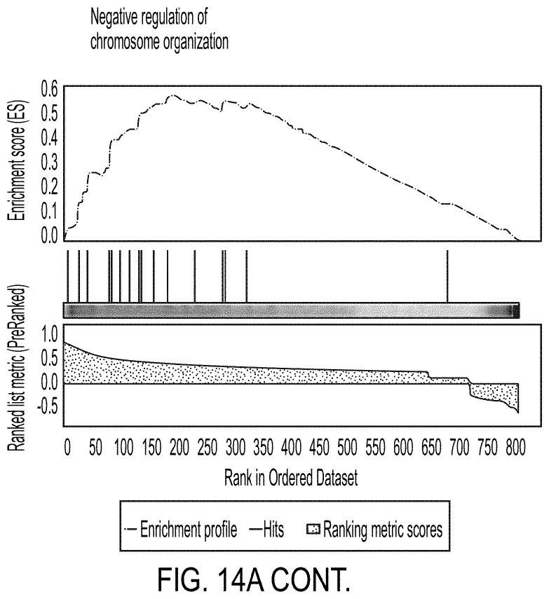

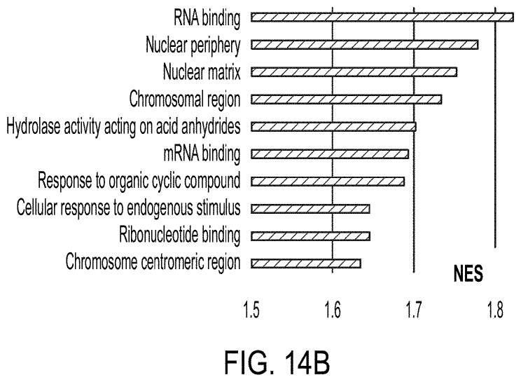

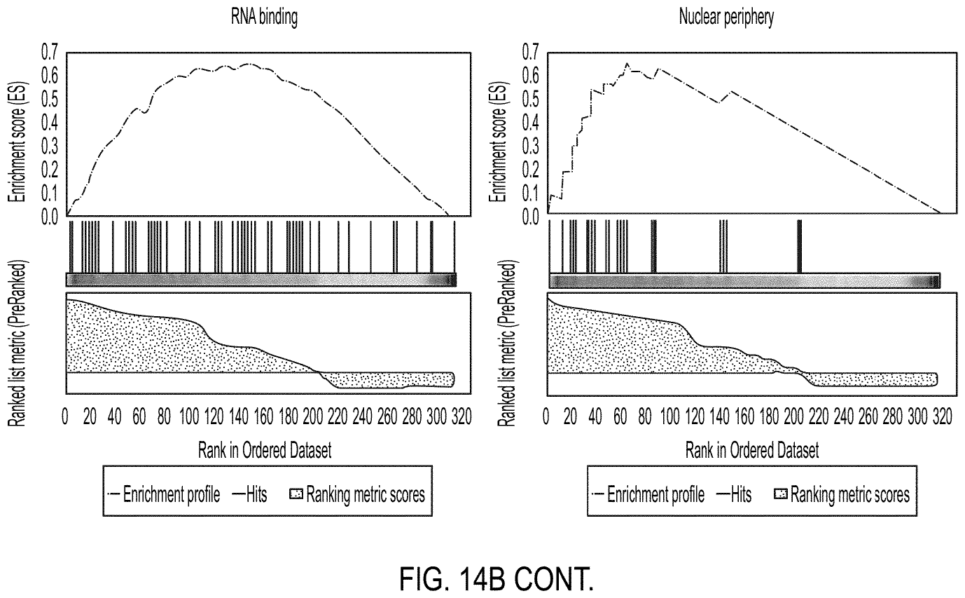

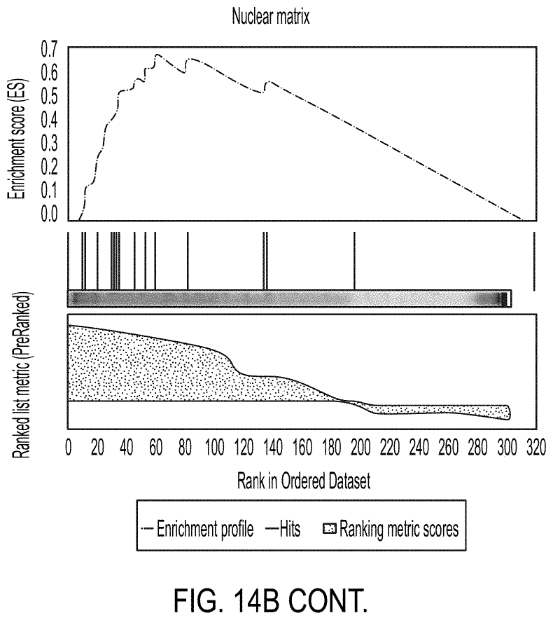

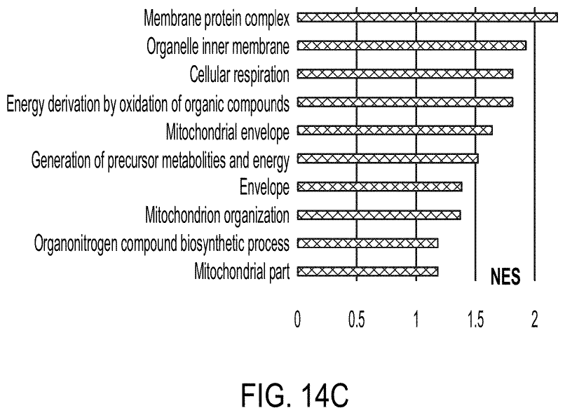

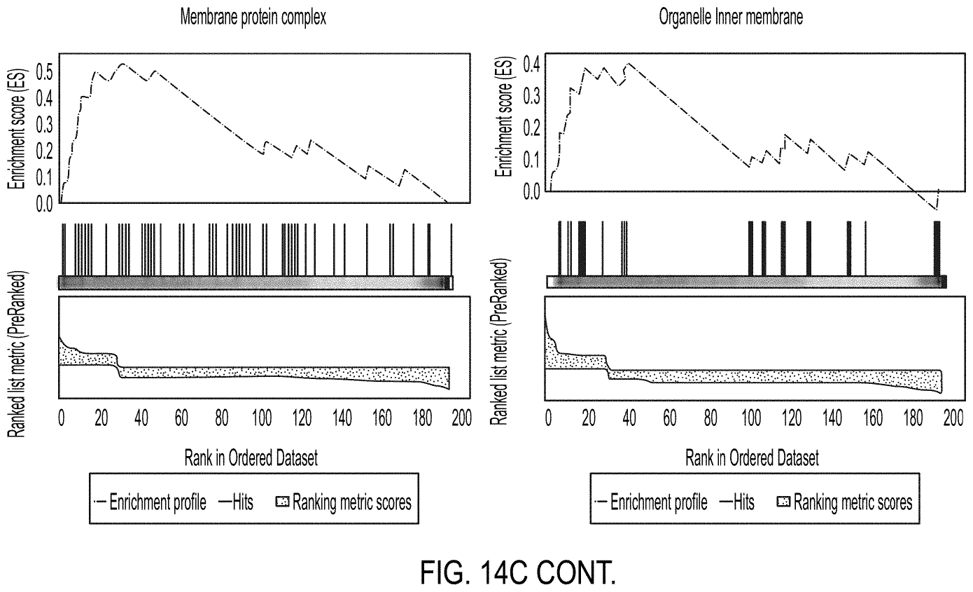

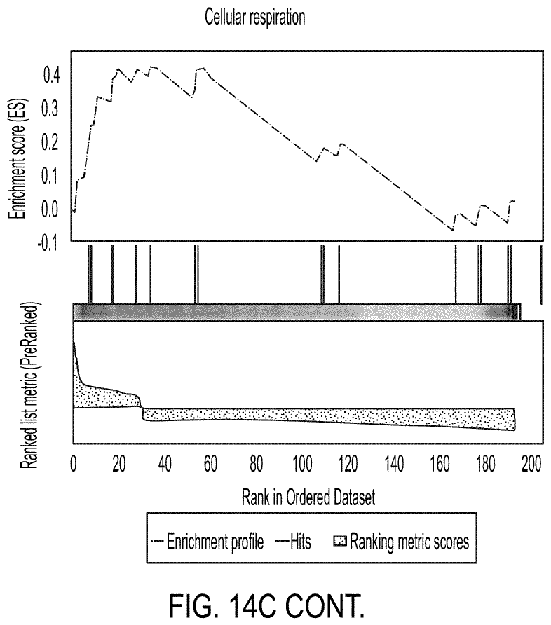

It was found that GO terms enriched in the cluster DEP_Human 0 mainly relate to protein translation and transportation (FIG. 6A). The top 10 enriched gene sets include biological processes such as protein localization to endoplasmic reticulum, protein targeting to membrane and nuclear transcribed mRNA catabolic process. The associated molecular function of structural constituent of ribosome and the cellular component activities such as ribosome and cytosolic part are also observed. When compared to one another, GSEA of cluster Human 0 shows overlap to a high degree in terms of the top enriched GO terms (FIG. 6B). Besides several shared features, co-translational protein targeting to membrane, cytosolic ribosome and translational initiation are also correlative activities during the translation. The results indicate that, most of the cells in cluster DEP_Human 0 and Human 0 are converting the information carried in mRNAs into proteins. Furthermore, we also observed similarities between cluster DEP_Human 1 and Human 1, both dominated by transcriptional processes such as ribonucleotide binding, double stranded DNA binding and mRNA metabolic process (FIGS. 14A and 14B). Here, the enrichment plot of one of the shared GO sets is shown, and the expression pattern of top-ranked marker gene (RPS7) that defines each cluster is also provided. The top 10 list of GO terms of the cluster DEP_Human 2 is shown in FIG. 14C, which does not present clear biological processes, but metabolic events are associated with this subset of cells since mitochondrial activities play the essential role in cell metabolism.

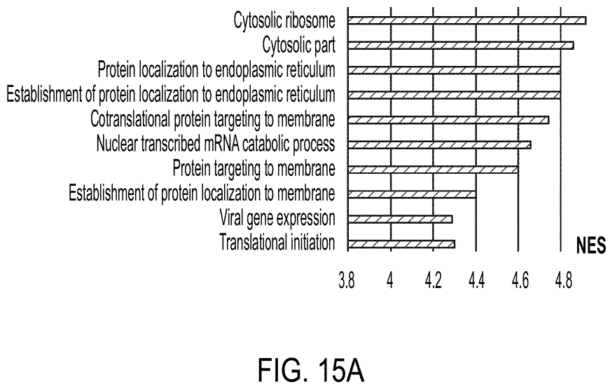

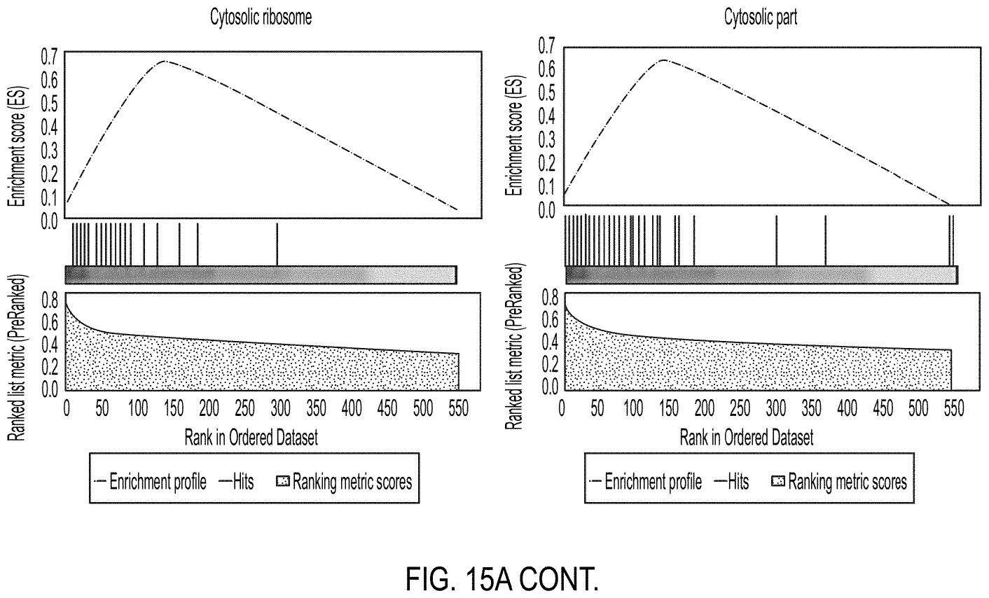

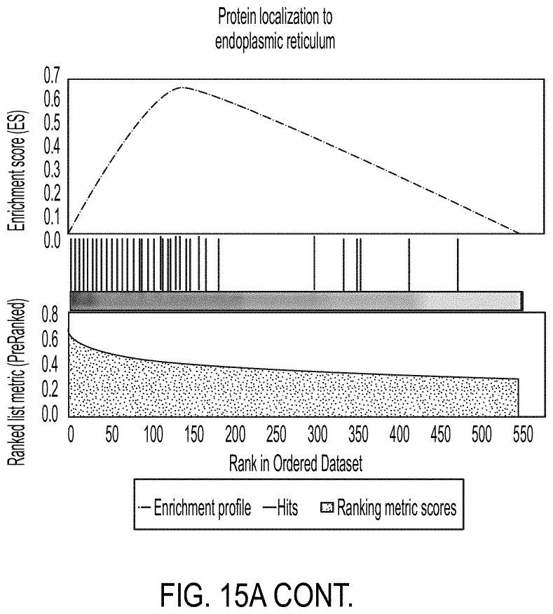

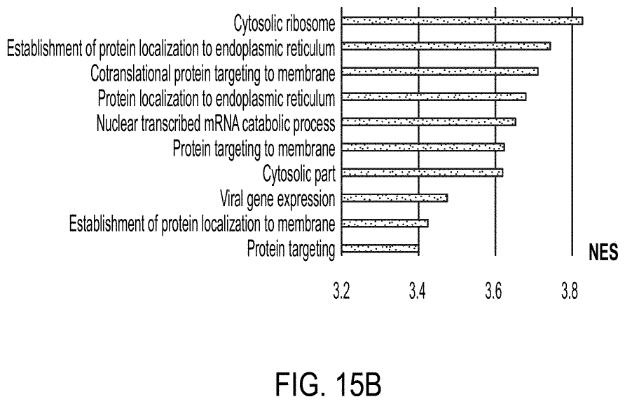

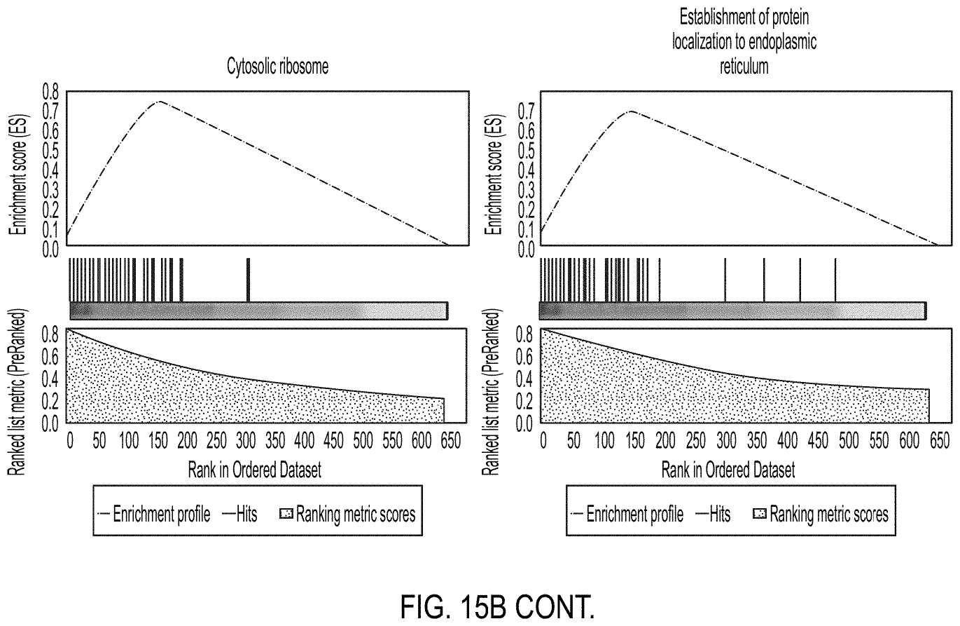

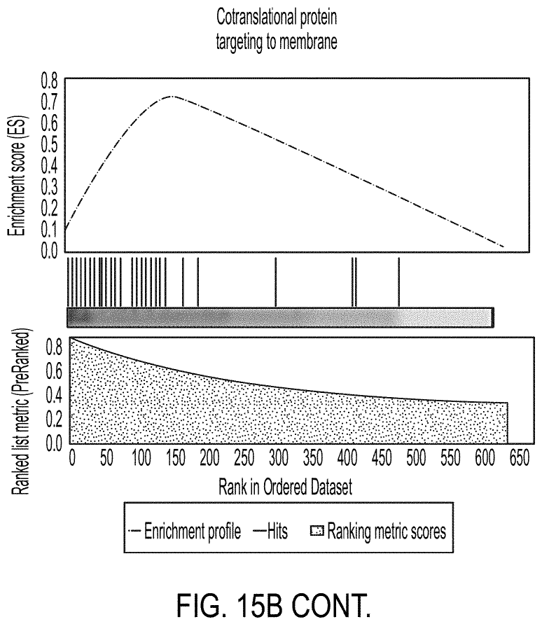

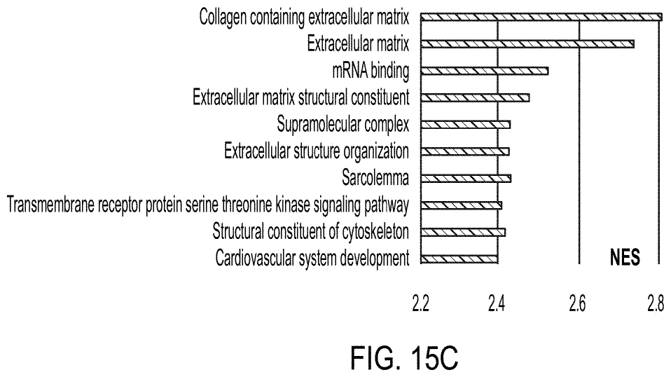

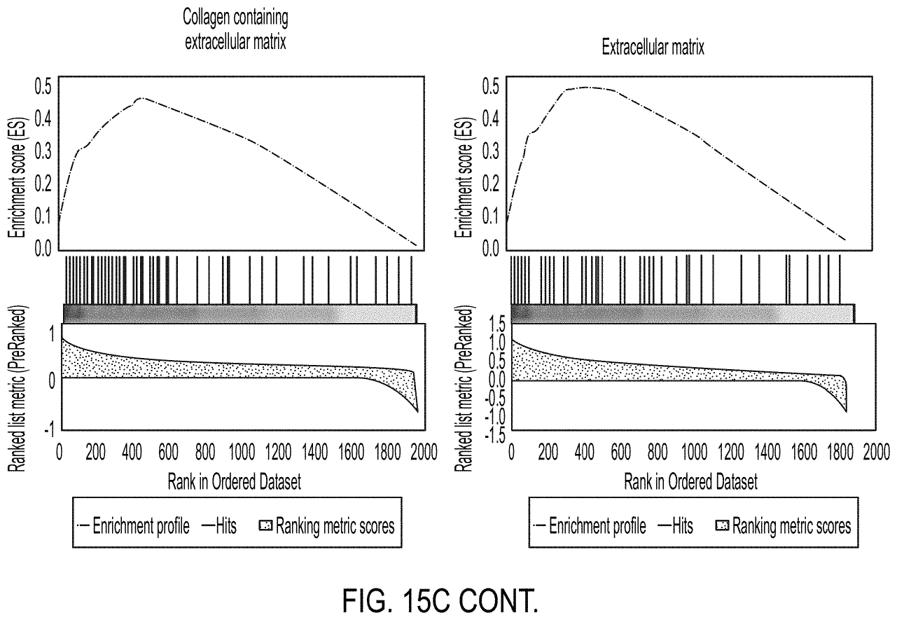

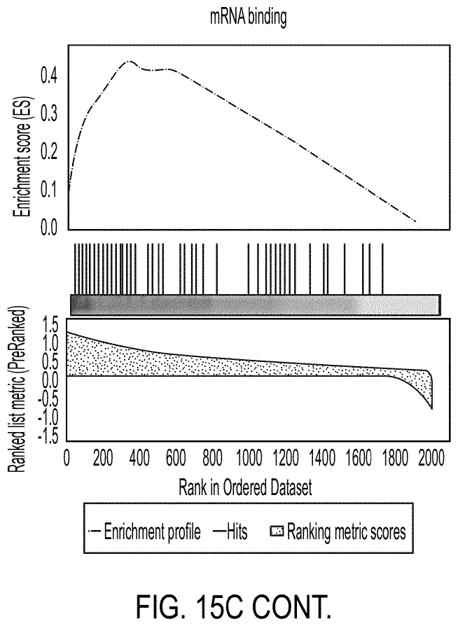

For the mouse cell clusters, DEP_Mouse 0 shows translational and ribosomal activities similar to that in DEP_Human 0 (FIG. 15A). Unsurprisingly, the cluster Mouse 0 generated from non-DEP device has similar enriched biological activities, in which 9 out of top 10 GO terms are consistent with that in DEP_Mouse 0 (FIG. 15B). Similarly, the cells in DEP_Mouse 1 and Mouse 1 have almost identical features as well (FIG. 6C, 6D). Activities significantly enriched in these two clusters are mitotic cell cycle and cell division, the process resulting in partitioning of components of a cell to form more daughter cells. Chromosomal components, the cellular structures in which genes perform functions such as DNA replication are also identified. The upregulated expression of gene Cenpa, which encodes centromere protein A that specify the mitotic behavior of chromosomes also verify these observations. In DEP_Mouse 2, the formation of extracellular structures is carried out, which can provide not only essential physical scaffolding for the cellular constituents but also initiate crucial biochemical and biomechanical cues required for tissue morphogenesis, differentiation and homeostasis (FIG. 15C).

Reassuringly, these findings highlight that the use of DEP trapping has no or little effect on the transcriptional profiles of single cells analyzed by scRNA-seq. Thus, it demonstrates that dTNT-seq is a valid approach to acquire biologically meaningful data from a large number of single-cell transcriptomes without alteration of cellular behaviors and data quality. Equally important, these data suggest that, even for genotypically homogeneous samples, scRNA-seq can distinguish transcriptional states that are mostly shown in transient cellular activities such as transcription, translation and metabolism.

### Conclusion

To democratize the use of scRNA-seq in biological and biomedical research and ultimately translate it for precision medicine and health management, efforts are still needed to further reduce cost, increase cell capture rate, improve ease of use, and portability. However, current scRNA-seq technologies such as 10× Genomics, DropSeq and SeqWell are all based on random passive encapsulation or pairing of cells and beads, which have a fundamental limit imposed by the Poisson statistics to prevent from further increasing cell-bead pairing efficiency. To address this challenge, dTNT-seq was developed, an active DEP-based trapping approach for single-cell transcriptome sequencing that allows for breaking the Poisson limit. By pre-aligning the two components of the dTNT device (an electroactive DEP 3,600-nanowell array and a larger-sized microwell array), it was demonstrated that a single-cell trapping rate of 91.84% with less than 2% doublets and an efficient transfer rate of 82%. Moreover, it is a particular superiority of our device that the compact configuration economizes the consumption of expensive DNA barcoded beads by reducing the number of nanowells required for generating the same number of single-cell transcriptome data points as compared to other microwell-based or droplet-based methods. In the validation of dTNT-seq, a stringent human-mouse species mixture experiment was conducted. The inventors recovered 1,155 HEK cells and 1,019 NIH3T3 cells from a single device containing as few as 3,600 nanowells. The inventors demonstrated a comparable performance with regards to the number of genes and transcripts detected per cell. Unsupervised clustering and GSEA analysis identified subtle differences of biological processes underlying the gene expression patterns and transcriptional states. Finally, through comparison with non-DEP microwell-based methods (i.e., scFTD-seq), we certified that the use of DEP has no or little impact on cellular states at the transcriptional level, evidenced by that the identified clusters from dTNT-seq and scFTD-seq shared highly consistent gene expressional clusters and GO pathways.

DEP trapping rate is a key index for obtaining enough single cells to perform downstream experiments and achieve high quality transcriptome profiling. To broaden the application of dTNT-seq, rigorous assessment of all operating parameters that determine single cell capture efficiency is critical. Current study investigated the effect of nanowell depth and suggested 10 μm nanowells could provide an optimal single-cell occupancy ratio with a neglectable doublet rate. Other factors, including conductivity of the surrounding medium, frequency and strength of the applied electric field, diameter of the nanowells, and flow rate of cell suspension have been evaluated experimentally. In addition, the dielectric properties of various biological cells, such as mouse lymphocytes and erythrocytes. human erythrocytes, normal and malignant white blood cells, and leukemia cells, have also been studied. Furthermore, besides 3T3 and HEK used in our study, DEP has been successfully applied to capture and manipulation of different types of cells including human monocytic-like cells (U937), lung cancer cells (NCIH1975), prostate cancer cells (DU145 and PC3), breast cancer cells (MCF-7) and HeLa cells at single cell level. Taken together, all these can help extend the use of dTNT-seq to a broad range of research areas.

Throughput is another important factor in single-cell analysis. To maximize the identification of transcripts, most of the scRNA-seq experiments target the analysis of several thousands of single cells per sample per run, considering the sequencing depth required per cell to yield biologically meaningful data. When too many cells are pooled in single sequencing lane, the depth per cell is much reduced and thereby the number of genes detectable per cell decreases substantially. Therefore, we devised and fabricated 3,600 nanowells for DEP trapping of single cells to validate the utility of dTNT-Seq, which is adequate for most applications. If needed, current device can be easily scaled up to tens of thousands of chambers for massively parallel single cell capture without significant changes in design. One of the potential concerns that may be encountered when increasing the number of nanowells per device is the electrothermal interference, including increased Joule heating and localized dielectric loss heating, which may affect the cell viability and change cellular behaviors. However, the problem can be mitigated to the greatest extent by dividing the DEP electrode array into sub-blocks while keeping the signal connection pad linked to a centralized control module.

The dTNT-seq approach can be further improved or expanded to other analyses unreachable by current technologies. First, we can maximize the capture efficiency for precious patient samples by connecting inlet and outlet together so that cells are re-circulating in the flow channel. Second, dissecting cellular crosstalk by sequencing physically interacting cell-cell pairs is of huge demand in the tumor ecosystem research and remains a challenge. Since cell pairs can be readily formed using DEP, we envision this DEP-based scRNA-seq method can be modified for studying cell-cell interactions in a highly controlled manner by independently operating a pair of DEP traps to capture heterotypic cells and followed by high-throughput transcriptome sequencing of cell-cell pairs. Examples are tumor and stromal cell interaction, tumor and macrophage interaction, and T-cell and dendritic cell interaction. Overall, this DEP-trapping-nanowell-transfer strategy is an enabling platform for the profiling of mRNA transcriptome from single cells or cell-cell pairs implicated broadly in basic or clinical biomedical research.

### Methods

### DEP Trapping Theory.

DEP is a phenomenon describing the directional movement of a dielectric particle in a nonuniform electric field. For a spherical particle with a radius of r, DEP force (F_{DEP}) acting on it can be calculated by

F_{DEP}=2πεₑrRe[K(2πf)]∇|Eₑ|², Equation 1

where ε, f and Eₑ represent the absolute permittivity, the activation frequency and the amplitude of the applied electric field, respectively. K is the polarization factor, and can be expressed as

$$K ( 2 π f ) = \frac{{ε}_{cell}^{⋆} - {ε}_{e}^{⋆}}{{ε}_{cell}^{⋆} + 2 {ε}_{e}^{⋆}} , \qquad Equation 2$$

### Here,

$${ε}^{⋆} = \text{?} + j \frac{σ}{2 π f} ( j = \sqrt{- 1} and σ is electrical conductivity ) .$$

$$\text{?} \text{indicates text missing or illegible when filed}$$

[symbol] and [symbol] are the complex electrical permittivity of the particle or cell and the culture medium, respectively. Re[K (2πf)] is the real part of the Clausius-Mossotti (CM) factor, which determines the polarity and thereby the direction of F_{DEF}, which can be adjusted by the conductivity of the surrounding medium and the frequency of the applied electric field. For a cell more polarizable than the surrounding medium (i.e., Re[K(2πf)]>0), it is attracted toward the maximum of electrical field gradient by positive DEP. In the case of a cell that is less polarizable (i.e., Re[K(2πf)]<0), it is repelled away from the high electric field gradient region by negative DEP. In our study, positive DEP force was employed to trap cells into microwells.

### Device Fabrication

The DEP nanowell chip was fabricated utilizing standard soft lithography and lift-off techniques. Glass slides (Thermo Scientific; 3″×1″×1 mm thick) were first cleaned by piranha solution (a 3:1 mixture of sulfuric acid and hydrogen peroxide) at 350° C. for 5 min, followed by deionized (DI) water rinsing and prebake. Before photoresist deposition, several drops of HMDS (Microchem) were coated to improve adhesion. A layer of AZ5214 (Microchem) that is capable of image reversal was then spun at 4000 rpm for 40 sec, resulting in about 1.5 μm film. After soft-bake at 110° C. on hotplate for 50 sec, an inverse pattern was created by exposure under UV illumination with a dose of about 100 mJ/cm² (EVG 620 Contact/Proximity Mask Aligner). Then, the chip was post baked at 120° C. for 2 min and the flood exposure is applied to make the unexposed areas soluble, with typical dose of more than 200 mJ/cm². Subsequently, the photoresist was developed using MF319 developer (Microchem) for 1 min. Before metal layer coating, the chip was treated with oxygen plasma for the removal of resist residues and contaminants from the surface. Afterwards, 20 nm thickness titanium and 100 nm thickness gold were successively deposited by thermal evaporation (Kurt Lesker EJ1800 Thin Film Deposition System). Finally, the rest of the sacrificial photoresist together with the covered metal was washed out with acetone. To fabricate the 10 μm depth nanowells layer on the gold electrodes, SU8 2010 (Microchem) was spread onto the substrate at 500 rpm for 5 sec then increased to 3500 rpm for 45 sec. The nanowell array pattern printed on a chromium photomask was precisely aligned with the patterned electrodes and the SU8 photoresist was exposed to UV light using MJB4 Mask Aligner (SUSS MicroTec) equipped with a long pass filter. After post-bake, the whole chip was developed and rinsed following the processing guideline, and a 5 min hard bake at 150° C. was incorporated into the process to cure the photoresist.

For the fabrication of the larger microwell layer, a normal workflow designed was followed for SU8 negative photoresist micromachining, but the entire process was modified and optimized for the specific application using PMMA substrate (Eplastics, Ridout Plastics, Inc.). Firstly, a 2 mm thickness PMMA was cut into 4.5 cm×2.3 cm rectangles by a computer numerical controlled laser cutter. Prior to applying resist, the substrate was cleaned with isopropyl alcohol (IPA) and DI water in ultrasonic bath and then pre-baked at 60° C. for 3 min. Secondly, SU8 2025 (Microchem) was spun coating at 500 rpm for 5 sec, followed by 1750 rpm for 45 sec to produce a 50 μm thick film. A build-up of photoresist on the edge of the substrate was removed by using a small stream of EBR PG (MicroChem) for the close contact with photomask. After soft bake at 60° C. for 2 min and then at 90° C. for 15 min, the substrate was exposed to UV light at dose of 170 mJ/cm². Directly after exposure, the chip was post baked at 60° C. for 2 min and then at 90° C. for 15 min, and subsequently underwent a relaxation step at 60° C. for 2 min. A 15 min development was performed using SU8 developer (Microchem), followed by a 10 sec IPA spray. Finally, the device inlet and outlet ports were drilled using laser cutter.

### Cells

NIH 3T3 mouse fibroblasts were used for the dTNT-seq validation of single cell capture and transfer efficiency. The cells were cultured in Dulbelcco's modified Eagle's medium (DMEM; Gibco), with glutamate and supplemented with 10% fetal bovine serum (Gibco) in a humidified incubator (37° C. in an atmosphere of 5% CO₂). Before use, cells were detached from the bottom of the culture flasks by applying 1 mL of Trypsin-EDTA (Sigma Aldrich) and incubate at 37° C. for 3 min. Cells were stained with a green fluorescent probe (CellTracker Blue CMAC, Invitrogen) following the manufacturer instructions. Briefly, the 3T3 cells were resuspended at a density of 2×10⁶ cells/mL in serum-free DMEM containing 5 μM/mL dye. After incubation at 37° C. in dark for 15 min, the labeled cells were washed twice in complete DMEM medium. For the species-mixture scRNA-seq assay, HEK cells were cultured in the same medium and stained with a red fluorescent dye (CellTracker Red CMTPX, Invitrogen) as described above. Equal numbers of 3T3 and HEK cells were mixed together before loading.

### DEP Buffer

Typically, culture medium has a high conductivity that can only induce a negative DEP response to mammalian cells. To produce positive DEP to trap cells, a low conductivity DEP buffer that was composed of 10 mM HEPES, 0.1 mM CaCl₂, 59 mM D-glucose and 236 mM sucrose was prepared. In addition, 2% w/v bovine serum albumin (BSA; Sigma Chemical Co.) was added to the DEP buffer to block nonspecific cell adhesion. The final conductivity of the buffer was measured by a conductivity meter (EC215, Hanna Instruments), and the averaged read was 272 μS/cm. Notably, the cell viability in this buffer was already verified by several reports.

### Device Assembly and Experimental Setup

Before assembling, each separate part of the dTNT device was exposed to 02 plasma to make the SU8 nanowells hydrophilic. The 100 μm-thickness PDMS gasket with central hollow rectangular cutout was first attached to the DEP nanowell chip. Then, the larger microwell array was vertically aligned on top of the DEP array utilizing our home-built manipulator (Thorlabs, Inc.) together with a microscope. The assembled device was fixed and clamped by two PMMA plates using spring-adjusted screws. Before use, ethanol was slowly flowed through the device to remove air bubbles in the SU8 nanowells. Thereafter, the device filled with 5% BSA in PBS was incubated at room temperature for 30 min. Subsequently, device was washed with DEP buffer prior to cell loading. During the experiments, the dTNT device was placed on the EVOS™ FL Auto Imaging System (Life Technologies) that integrates a fully automated and motorized X/Y scanning stage. The system was also used to monitor and image the whole trapping process. A function generator (SDG1000X; Siglent) was used to induce a sinusoidal electric potential. A 1 mL syringe (BD) connected with the device outlet was precisely controlled by a syringe pump (Fusion200, Chemyx Inc.).

Microfluidic Operation of dTNT-seq

Prior to the experiment, 250 μl (about 40,000) of the original beads was washed three times with PBS and resuspended in 50 μl of PBS; cell suspension was centrifuged at 300 g for 5 min and the culture medium was changed with the low conductivity DEP buffer; 500 μl of the freeze-thaw lysis buffer composed of 100 mM Tris (pH 7.5), 10 mM EDTA, 1M NaCl, 5 μM DTT, 0.4 U/mL Lucigen RNase was prepared freshly and kept on the ice. The bench and microscope were carefully cleaned with RNAse away and 70% alcohol. A total of 20 μl cell suspension with a density of 2×10⁶ cells/ml was pipetted on the inlet reservoir and withdrawn into the dTNT device channel by the syringe connected to the device outlet. According to the real-time imaging during the DEP trapping, once single cell capture achieved the desired rate, more DEP buffer was added to remove excessive cells. Then, the whole device was turned upside down while keeping the DEP voltage on, after which the DEP was stopped and captured cells were allowed to drop into larger microwells by gravity. The similar procedure for loading barcoded beads was then performed and excessive beads were washed out with PBS. Next, 200 μl of lysis buffer was loaded and 500 μl fluorinated oil (Fluorinert FC40) was introduced into the device to seal the microwells.

Device Preparation and Microfluidic Operation of scFTD-seq

The detailed materials and methods for preparing and operating the scFTD-seq device have been described in our previous report. Briefly, the device consisted of a microwell array layer and a microfluidic channel layer, both of which were made by casting PDMS over the SU8 master wafers followed by degassing and curing at 80° C. for 6-8 hours. After curing, PDMS was peeled off, and the two layers were cut to proper sizes and then plasma-bonded to assemble onto a glass slide. Prior to cell loading, device was pressurized to remove air bubbles inside the microwells using a manually operated syringe with outlet closed, and then primed for 1 h at room temperature with 1% BSA in PBS. A total of 50 μl cell suspension with a density of 2×10⁶ cells/ml was pipetted on the inlet reservoir and withdrawn into the device. Finally, barcoded beads, lysis buffer and fluorinated oil were loaded sequentially, similar as that in dTNT-seq.

Cell Lyse and mRNA Capture for dTNT-Seq and scFTD-Seq

After cell and beads loading, the device was placed in a petri dish and exposed to three cycles of freeze-and-thaw, each of which included freezing at −80° C. freezer or dry ice for 10 min followed by thawing at room temperature for 10 min. To capture mRNA onto beads, the dTNT device was incubated for 1 h inside an aluminum foil covered wet chamber. After incubation, the device was inverted back and the beads were retrieved by 6×saline-sodium citrate (SSC) buffer flushing. Finally, collected beads were washed twice with 6×SSC buffer and then proceeded to the reverse transcription step.

### Library Preparation and Sequencing

For both dTNT-seq and scFTD-seq, library preparations were performed as described in the DropSeq and SeqWell protocol (version 3.1, http://mccarrolllab.com/dropseq/). Briefly, the captured mRNA was reverse-transcribed using Maxima H Minus reverse transcriptase (Thermo Fisher) with a custom template switching oligo. Then, the beads coated with cDNA were treated using Exonuclease I (Exo I, NEB) for 1 h at 37° C. with rotation to chew away any unbound mRNA capture probes. The cDNA was then amplified using a 13-cycle PCR whole transcriptome amplification, followed by purification of the cDNA library using Ampure XP beads (Beckman Coulter) at 0.6 ratio. Table 2 lists the sequence of the beads and all the primers used in library preparation. The quality of the amplified DNA was assessed by Bioanalyzer (Agilent Inc.) using high sensitivity chip. After a standard Nextera tagmentation, PCR reactions (Nextera XT, Illumina), another round of purification and high sensitivity Bioanalyzer test, the libraries were sequenced on HiSeq4000 (Illumina) at medium depth (average of 20 k to 40 k reads/cell) with 4 samples pooled into one sequencing lane.

TABLE 2
The sequence of the beads and primers
used in library preparation.

| Name | Usage | Company | Sequence |
|---|---|---|---|
| Barcoded | mRNA | ChemGenes | 5′-Bead-Linker-- |
| Beads | capture | (Customized) | TTTTTTTAAGCAGTGGTATCAACGC |
| | | | AGAGTACJJJJJJJJJJJJNN |
| | | | NNNNNNTTTTTTTTTTTTT |
| | | | TTTTTTTTTTTTTTTTT-3′ |
| | | | SEQ ID NO: 1 |
| Template | Reverse | IDT | 5′- |
| Switch | transcrip- | (Customized) | AAGCAGTGGTATCAACG |
| Oligo | tion | | CAGAGTGAATrGrGrG-3′ |
| | | | SEQ ID NO: 2 |
| SMART | PCR | IDT | 5′-AAGCAGTGGTATCAACGCAGAGT-3′ |
| PCR Primer | | (Customized) | SEQ ID NO: 3 |
| New-P5- | Library | IDT | 5′-AATGATACGGCGACCACCGAG |
| SMART | preparation | (Customized) | ATCTACACGCCTGTCCGCGGA |
| PCR hybrid | | | AGCAGTGGTATCAACGCAGA |
| oligo | | | GT*A*C-3′ SEQ ID NO: 4 |
| Custom | Sequence | IDT | 5′-GCCTGTCCGCGGAAGCAGTG |
| Read 1 | primer | (Customized) | GTATCAACGCAG |
| Primer | | | AGTAC-3′ SEQ ID NO: 5 |

### Transcriptome Alignment and Data Analysis

Original raw reads were transformed into digital gene expression matrix (DGE) following Drop-seq core computational protocol (V2.0.0). Briefly, the 5′ adapter and 3′ poly A tails were detected and trimmed to remove adapter sequence, and the cell barcode and UMI was organized and matched to each gene in each cell. The paired reads were then aligned to the human-mouse mix reference genome (hg19_mm10) using STAR v2.5.2b. DGE was generated for the cells with over 10 000 reads per cell and 2748 cells were identified. The Seurat package (V3.0) in R (V3.6) was used to perform all the data analysis. For the species assignment, cells with <500 genes, <2000 transcripts were considered low-quality and filtered out, and cells with >90% transcript purity were considered as belonging to the human or mouse species. For the unsupervised clustering, the quality control criteria utilized to filter cells include: 1) gene expression counts between 200 and 5,000 genes; 2) less than 10% expression of the mitochondrial genes (Figure S5b&c). After data filtering, 2,570 cells were obtained for clustering analysis for dTNT-seq, and 1595 cells were left for scFTD-seq. A global-scaling normalization and a linear transformation (‘scaling’) were applied as a standard pre-processing step prior to performing the PCA. To reduce the uncertain of identifying the true dimensionality of the dataset, both the JackStraw procedure and the Elbow method were utilized to determine the top principal components that will be included in the clustering.

### Gene Set Enrichment Analysis

GSEA software was used to analyze the gene expression pattern of each cluster. Here, differentially expressed genes distinguishing each cluster from other clusters and the corresponding fold change values were loaded into the GSEAPreranked. The complete collection of Gene Ontology (GO) including biological process, cellular component and molecular function were used as the annotated gene sets. For mouse clusters, the genes were converted from the GO gene sets to a target Mouse Gene Symbol Remapping gene sets using Chip2Chip analysis.

### Example 2

The successful demonstration of dTNT-seq verifies that “Single cell trapping by DEP—transfer to larger microwells-load barcoded beads-capture transcriptome” could be a reliable pathway of conducting double-sub-Poisson active manipulation of single cells for scRNA-seq. We envision the dTNT-seq device can be modified for more sophisticated and demanding applications from three aspects.

### Design Adjacent Electrode Pairs to Capture Two or More Types of Cells to Study Cell-Cell Interactions at Single Cell Level

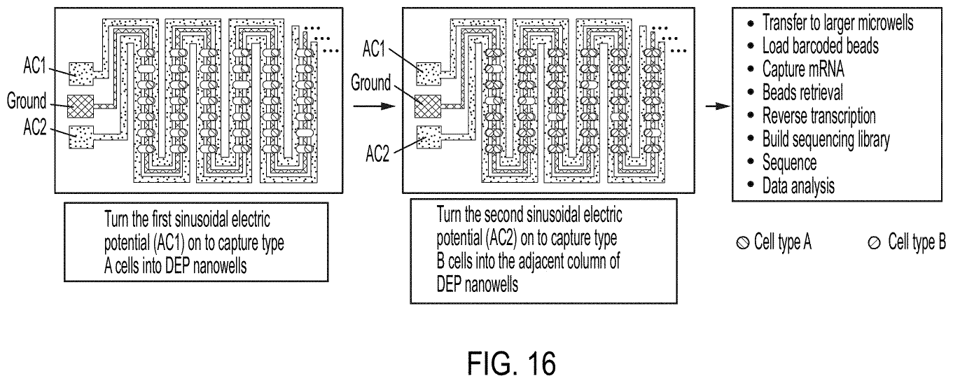

Dissecting cellular crosstalk by sequencing physically interacting cell—cell pairs is of huge demand in the tumor ecosystem research and remains a challenge. We anticipate to add adjacent DEP trap units on the current DEP nanowell array layer so that two or more types of cells can be captured. FIG. 16 diagrams the design pattern and workflow of pairing two types of cells for studying cell-to-cell interactions through scRNA-seq. After assembling the two separate layers into an integrated device and loading the type A cells, turn the first sinusoidal electric potential (AC1) on to capture type A cells into DEP nanowells at right side. Then, load the type B cells and capture them into left side DEP nanowells by turning the second sinusoidal electric potential (AC2) on. Thereafter, the whole device can be flipped to transfer the captured cells into the larger microwells below, followed by beads loading, mRNA capture, etc. To study the interactions of more types of cells, more DEP trapping units can be designed to sequentially capture each type of cells before transferring them into larger microwells.

### Design the “Roof DEP Nanowell Array” Device to Directly Capture Single Cells on Top

Current dTNT-seq is a relatively complex integrated device, and the complexity mainly stemmed from the “transfer” process. A design of a reversed dTNT-seq device, in which the DEP nanowell array is on the top and the larger microwell array is on the bottom after the initial assembly is contemplated herein. As the schematic illustration shown in FIG. 17A, after loading type A cells into the flow channel (not shown), turn AC1 on to directly capture them on the “roof” DEP nanowells. Then, turn AC1 off to release the type A cells and let them drop into the bottom larger microwells by gravity. The DEP nanowells are available at this time point to capture other cells. If the studying of cell-to-cell interactions is desired, load type B cells, capture them by turning AC2 on, and release them into larger microwells in a similar manner. When the cell trapping is finished, load barcoded beads to capture mRNA for the downstream sequencing and analyses. FIG. 17B shows the cross-sectional illustration of the whole process.

### Design Addressable “Roof DEP Nanowell Array” Device to Enable Flexible Manipulation of Cells of Interest

On the basis of the functions described above, a design is contemplated having a control circuit to enable each DEP nanowell programmable and addressable so that single cells at any step, either during the trapping process or already transferred into the bottom larger microwells, can be manipulated flexibly. As shown in FIG. 18, after capturing two types of cells, for example, T cells (Type A) and tumor cells (Type B), through “Roof DEP nanowell array” and transferring them into the bottom larger microwell #1, the cell-to-cell interactions can be observed or measured on a real-time imaging microscopy system. If cells in any microwells exhibit specific states or functions, for example, T cells show highly cytotoxic tumor killing capability, we term them as cells of interest (COI). The exact positions of COI can be located, and the corresponding DEP electrical field can be turned on using the programmable control system. Then, the selected COI can be captured back on the top DEP nanowells again and transferred into a new larger microwell array (#2) for further in-depth analyses.

### EQUIVALENTS

Although preferred embodiments of the invention have been described using specific terms, such description is for illustrative purposes only, and it is to be understood that changes and variations may be made without departing from the spirit or scope of the following claims.

### INCORPORATION BY REFERENCE

The entire contents of all patents, published patent applications, and other references cited herein are hereby expressly incorporated herein in their entireties by reference.

1. A method for single-cell RNA sequencing comprising:
aligning a microwell array on top of a dielectrophoresis (DEP) single-cell-trapping nanowell array;
loading a plurality of cells into the nanowell;
applying electricity to the nanowell array to trap a quanta of cells equal to a quanta of electrode pairs in at least one nanowell of the nanowell array;
discontinuing electricity to the nanowell array in order to transfer the loaded cells from the nanowells to the microwells;
loading a plurality of barcoded beads into the microwells so that a single bead occupies each cell-loaded microwell;
capturing RNA from the cells and retrieving the RNA-loaded beads; and,
sequencing the captured RNA.

2. The method of claim 1, wherein the microwell array comprises wells having a 50 μm diameter.

3. The method of claim 1, wherein the nanowells have a diameter selected from: 10 μm, 15 μm and 20 μm.

4. The method of claim 1, wherein the RNA is sequenced using one or more techniques comprising PCR.

5. The method of claim 1, wherein the cells are loaded into the nanowells by applying a first alternating electrical potential.

6. The method of claim 1, further comprising loading a plurality of a second cell type into the nanowells.

7. The method of claim 6 wherein the second cell type is loaded with a second alternating electrical potential.

8. The method of claim 1, further comprising:
inverting the aligned arrays so that the microwell array is beneath the nanowell array.

9. A DEP-trapping-nanowell-transfer (dTNT) system comprising:
a single cell trapping nanowell array, and
a microwell array pre-aligned on top of the nanowell array, wherein the microwell array is aligned with a microaligner device.

10. The dTNT system of claim 9, wherein the single-cell trapping nanowell array comprises wells having a dimeter selected from: 10 μm, 15 μm and 20 μm.

11. The dTNT system of claim 9, wherein the microaligner device is adapted and configured to align the wells of the nanowell array with the wells of the microwell array.
